(19) United States
(12) Patent Application Publication
(10) Pub. No.: US 2024/0408553 A1
(43) Pub. Date: Dec. 12, 2024

(54) HOLLOW FIBER MEMBRANE, HOLLOW FIBER MEMBRANE MODULE AND VESICLE-CONTAINING SOLUTION

(71) Applicant: Toray Industries, Inc., Tokyo (JP)

(72) Inventors: Kojiro Naito, Otsu-shi, Shiga (JP); Akihiro Hayashi, Otsu-shi, Shiga (JP); Yoshiyuki Ueno, Otsu-shi, Shiga (JP); Mie Kaino, Kamakura-shi, Kanagawa (JP)

(73) Assignee: Toray Industries, Inc., Tokyo (JP)

(21) Appl. No.: 18/699,346

(22) PCT Filed: Oct. 21, 2022
(86) PCT No.: PCT/JP2022/039278

§ 371 Date: Apr. 8, 2024

(30) Foreign Application Priority Data
Oct. 28, 2021 (JP) 2021-176308

(51) Int. Cl. B01D 69/02; A61M 1/34; B01D 67/00; B01D 69/08; B01D 71/48; B01D 71/68

(52) U.S. Cl. CPC B01D 69/02; A61M 1/3496; B01D 67/0016; B01D 67/0088; B01D 69/087; B01D 71/48; B01D 71/68; B01D 2325/0231; B01D 2325/02834; B01D 2325/04; B01D 2325/36

(57) ABSTRACT

A hollow fiber membrane, hollow fiber membrane module and vesicle-containing solution that includes a hollow fiber membrane and a hollow fiber membrane module capable of efficiently removing extracellular vesicles (vesicles) from a liquid, particularly from blood. One aspect of the hollow fiber membrane for achieving the above object is a hollow fiber membrane, in which particles having a particle size of 0.15 μm have a permeability of 50% or more and 100% or less, and an average pore size of an inner surface of the hollow fiber membrane is 0.50 μm or more and 3.00 μm or less.

### TECHNICAL FIELD

This disclosure relates to a hollow fiber membrane, a hollow fiber membrane module, and a vesicle-containing solution suitable for blood purification such as plasma separation.

### BACKGROUND

A treatment method by correcting a body fluid and removing a biotoxin is called blood purification. Blood purification includes hemodialysis, plasma exchange therapy, adsorption therapy and the like. The plasma exchange therapy is a treatment in which blood is separated into large-sized hemocyte components such as white hemocytes, red hemocytes, and platelets and plasma components including plasma and plasma proteins, and the separated plasma components are discarded and replaced with the same amount of fresh frozen plasma, or are secondarily processed to discard pathogenic substances and return them to the body. In this plasma exchange therapy, a porous hollow fiber membrane has been applied to plasma separation applications for separating plasma components from blood.

As described above, in the porous separation membrane for plasma separation, the pore size and the permeability of the substance are important from the viewpoint of separating the hemocyte component and the pathogenic substance contained in the plasma. In particular, it is required that platelets of about 3 μm, which is the smallest of hemocyte components, do not permeate. On the other hand, it is also required to permeate lipoproteins such as LDL cholesterol.

Other required characteristics include biocompatibility such as non-adsorption of proteins and hemocyte components and non-activation of complement. In a porous separation membrane made of a hydrophobic polymer, the membrane surface is coated with a hydrophilic polymer to impart hydrophilicity.

In addition, Theranostics 2018, Vol. 8, Issue 12,3348 reports that extracellular vesicles (hereinafter, it may be simply referred to as a “vesicle”) produced in patients with sepsis are not mere blood markers, but contain and carry messenger RNA (mRNA), thereby inducing inflammation in the whole body. Therefore, there is also a possibility that extracellular vesicles become targets of treatment and examination.

Japanese Translation of PCT International Application Publication No. 2009-542424 discloses a method of producing an asymmetric hollow fiber membrane made of a polysulfone-based polymer as a plasma separation membrane.

Japanese Patent Laid-open Publication No. 2017-56407 discloses a porous hollow fiber membrane for plasma separation including a polyolefin-type resin having a porous structure molded by a melt drawing opening pore method and a higher fatty acid metal salt.

Japanese Translation of PCT International Application Publication No. 2021-519421 discloses a method of removing extracellular vesicles from blood using a polyethersulfone hollow fiber membrane having a large porosity.

However, Japanese Translation of PCT International Application Publication No. 2009-542424 and Japanese Patent Laid-open Publication No. 2017-56407 merely disclose that a pore size may be designed within a range in which a hemocyte component does not permeate, and do not mention removing extracellular vesicles and the like from blood. In addition, the pore size indicates the maximum pore size measured by a bubble point method or the like, and the permeability of particulate substances such as extracellular vesicles is not sufficiently shown.

Japanese Translation of PCT International Application Publication No. 2021-519421 discloses a method of removing extracellular vesicles having a size lower than the porosity of a hollow fiber membrane, and shows that the fractionation performance of the hollow fiber membrane is different from that of the apparent pore size.

In general, a porous membrane is sieved according to a smaller one of pore sizes on a surface of a separation membrane, and therefore a pore size observed with a scanning electron microscope (SEM) or the like is used as an index of fractionation performance. However, depending on the separation membrane, the pore inside the membrane is smaller than that of the membrane surface, and sieving may be performed at that portion. That is, the pore size of the surface of the separation membrane does not indicate the fractionation performance of the separation membrane. On the other hand, a bubble point method is used as a method of representing the pore size of the separation membrane, but in the industrial standard (JIS), the bubble point method is used as a method [of evaluating the maximum pore size. Therefore, the pore size measured by the present method also does not represent fractionation characteristics.

Thus, there is a need for a hollow fiber membrane that efficiently removes extracellular vesicles (vesicles) having a size of about 40 nm to 1 μm present in a liquid as disclosed herein.

It could therefore be helpful to provide a hollow fiber membrane capable of efficiently removing extracellular vesicles (vesicles) from a liquid, particularly from blood.

### SUMMARY

As a result of intensive studies, we have found that the above problems can be solved by focusing on the particle permeability and the surface pore size of the hollow fiber membrane and adopting a predetermined hollow fiber membrane structure.

We thus provide:
[1] A hollow fiber membrane, wherein particles having a particle size of 0.15 μm have a permeability of 50% or more and 100% or less, and an average pore size of an inner surface of the hollow fiber membrane is 0.50 μm or more and 3.00 μm or less.
[2] The hollow fiber membrane according to [1], wherein a thickness of a dense layer in a cross section of the hollow fiber membrane is 1.0 μm or more and 10.0 μm or less.
[3] The hollow fiber membrane according to [1] or [2], wherein an average pore size of an outer surface of the hollow fiber membrane is 0.50 μm or more and 10.00 μm or less.
[4] The hollow fiber membrane according to any one of [1] to [3], wherein the hollow fiber membrane has an inner diameter of 150 μm or more and 500 μm or less and a membrane thickness of 20 μm or more and 100 μm or less.
[5] The hollow fiber membrane according to any one of [1] to [4], containing a polysulfone-based polymer.
[6] The hollow fiber membrane according to any one of [1] to [5], containing a hydrophilic polymer, wherein a content of the hydrophilic polymer is 5.0 mass % or more and 15.0 mass % or less.
[7] The hollow fiber membrane according to any one of [1] to [6], wherein at least one of the hydrophilic polymers is a hydrophilic polymer containing a monocarboxylic acid vinyl ester unit as a repeating unit.
[8] A hollow fiber membrane, which has permeability of a vesicle detectable by antibodies of phosphatidylserine and CD9 is 50% or more and 100% or less, and is used for separating the vesicle from a biological component.
[9] The hollow fiber membrane according to any one of [1] to [8], which is used for blood purification.

[10] A hollow fiber membrane module containing the hollow fiber membrane according to any one of [1] to [8].

[11] The hollow fiber membrane module according to [10], which is used for blood purification.

[12] A vesicle-containing solution purified by the hollow fiber membrane module according to or [11].

The hollow fiber membrane can efficiently remove extracellular vesicles (vesicles) in a liquid, particularly in blood.

### BRIEF DESCRIPTION OF THE DRAWINGS

FIG. 1 is a view of a cross section of a hollow fiber membrane produced by a method in Example 1 observed at a magnification of 2000 using a scanning electron microscope (SEM).

FIG. 2 is a view after binarization processing is performed on the SEM image of FIG. 1.

FIG. 3 is a view obtained by performing image analysis on FIG. 2 and deleting a pore having a pore size of 0.5 μm or less.

FIG. 4 is a view of an inner surface of a hollow fiber membrane produced by the method of Example 1 observed at a magnification of 1500 using a SEM.

FIG. 5 is a view of an outer surface of a hollow fiber membrane produced by the method of Example 1 observed at a magnification of 3000 using a SEM.

FIG. 6 is a schematic view of measurement of vesicle permeability.

### DESCRIPTION OF REFERENCE SIGNS

1: Example of thickness of dense layer
2: Tangent line drawn on outer surface
3: Perpendicular line to tangent line drawn on outer surface
4: Human serum pool
5: Circulation pump
5′: Filtration pump
6: Hollow fiber membrane module
7: Thermostatic bath
11, 12, 13: Outer surface side
21, 22, 23: Inner surface side

### DETAILED DESCRIPTION

Hereinafter, preferred examples of the hollow fiber membrane, the hollow fiber membrane module, and the vesicle-containing solution purified by the hollow fiber membrane module will be described in detail. However, our membrane, module and solution are not limited to the following examples, and various modifications can be made according to the purpose and the use.

### Hollow Fiber Membrane

In a hollow fiber membrane (hereinafter, it may be simply referred to as a “separation membrane”), particles having a particle size of 0.15 μm have a permeability of 50% or more and 100% or less, and an average pore size of an inner surface of the hollow fiber membrane is 0.50 μm or more and 3.00 μm or less. In the measurement of the particle permeability, polystyrene latex particles are used as described in “(2) Measurement of particle permeability” described later.

Since the hollow fiber membrane has a structure in which the vesicle can permeate but a hemocyte component does not permeate, the vesicle in the blood can be selectively removed. The vesicles have a distribution from 40 nm to 1 μm in diameter, but are flexible due to a lipid membrane structure thereof. Therefore, for example, even a vesicle having a diameter of 1 μm can transmit a pore smaller than 1 μm. The inventors have found that in the design of the hollow fiber membrane having a desired vesicle permeability, a particle permeability with a rigid particle size of 0.15 μm is an effective indicator. That is, to obtain desired vesicle permeability, the hollow fiber membrane has a permeability of particles having a particle size of 0.15 μm of 50% or more and 100% or less.

Examples of the method of setting the particle permeability within the above range include a method of adjusting a surface pore size of the hollow fiber membrane, a membrane thickness, and a thickness of a dense layer.

In addition, a liquid containing vesicles usually contains cells that produce vesicles and/or hemocyte components at the same time. The sizes of the hemocyte components are white hemocytes (10 μm or more), red hemocytes (about 7 μm), and platelets (about 3 μm), respectively. Since these hemocyte components are softly deformed, the size of the particles that can be blocked by the separation membrane is preferably smaller than 2 μm.

On the other hand, examples of the extracellular vesicles (vesicles) present in blood include exosomes (40 to 120 nm), microvesicles (50 to 1000 nm), and apoptotic bodies (500 to 2000 nm). Among them, since exosomes and microvesicles are said to be involved in communication between cells, it is required to remove exosomes and microvesicles in pathological conditions such as sepsis. From such a viewpoint, the particle permeability of the separation membrane having a particle size of 0.15 μm is preferably 70% or more, more preferably 80% or more, and still more preferably 85% or more. The upper limit of measurement of the particle permeability for a particle size of 0.15 μm is 100%, and therefore the particle permeability is 100% or less. The particle permeability with a particle size of 0.15 μm means a ratio at which particles with a particle size of 0.15 μm permeate.

From the viewpoint of removing microvesicles, the particle permeability of a particle size of 0.20 μm is preferably 50% or more, more preferably 55% or more, and still more preferably 60% or more. The particle permeability with a particle size of 0.20 μm means a ratio at which particles with a particle size of 0.20 μm pass. The upper limit of the particle permeability with a particle size of 0.20 μm is not particularly limited, and is preferably as large as possible. The upper limit of measurement, 100%, is ideal, but about 95% is sufficient.

In the hollow fiber membrane, an average pore size of an inner surface of the hollow fiber membrane is 0.50 μm or more and 3.00 μm or less. By setting the average pore size of the inner surface of the hollow fiber membrane within the above range, it is possible to prevent hemocyte components such as platelets from entering the hollow fiber. By preventing the entry of the hemocyte component into the hollow fiber, stimulation to hemocytes is reduced, blood compatibility is improved, and the vesicle can be efficiently separated from the liquid by suppressing clogging of the hemocyte component and the like. From the viewpoint of further improving the vesicle permeability and the water permeability, the average pore size of the inner surface of the hollow fiber membrane is preferably 0.80 μm or more and more preferably 0.90 μm or more. On the other hand, the average pore size of the inner surface of the hollow fiber membrane is preferably 2.50 μm or less and more preferably 2.00 μm or less, from the viewpoint of further preventing the entry of the hemocyte component and further increasing the strength of the hollow fiber membrane.

Examples of the method of setting the average pore size of the inner surface of the hollow fiber membrane within the above range include a method of adjusting the concentration of the main component (for example, polysulfone) constituting the hollow fiber membrane in the spinning dope solution at the time of spinning, and a method of adjusting the coagulation value of the injection liquid (core solution) to form the hollow portion. The coagulation value represents the added mass of the injection liquid at the time when the injection liquid is added little by little to 50 g of the solution having a concentration of the main component constituting the hollow fiber membrane of 1 mass % and the system becomes cloudy.

In the hollow fiber membrane, the average pore size of the outer surface of the hollow fiber membrane is preferably 0.50 μm or more and 10.00 μm or less. From the viewpoint of separation performance and water permeability, the average pore size of the outer surface of the hollow fiber membrane is more preferably 0.60 μm or more. From the viewpoint of the strength of the separation membrane, the average pore size of the outer surface of the hollow fiber membrane is more preferably 5.00 μm or less, still more preferably 3.00 μm or less, and particularly preferably 2.00 μm or less.

Examples of the method of setting the average pore size of the outer surface of the hollow fiber membrane within the above range include a method of adjusting the concentration of the anti-solvent vapor with respect to the main component constituting the hollow fiber membrane in a dry section during spinning described later, and a method of adjusting the spinneret temperature during spinning.

In the hollow fiber membrane, from the viewpoint of ease of production of the separation membrane, the average pore size of the outer surface of the hollow fiber membrane is more preferably smaller than the average pore size of the inner surface of the hollow fiber membrane. The position of the dense layer in the hollow fiber membrane is optional, and the dense layer may be located closer to either the inner surface or the outer surface. The thickness of the dense layer refers to the thickness of a region where no pore having a pore size of 0.5 μm or more is present when the cross section of the hollow fiber membrane is observed by a method described later. Hereinafter, in the hollow fiber membrane, the surface closer to the dense layer is referred to as “surface 1”, and the surface farther from the dense layer is referred to as “surface 2”. However, when a plurality of dense layers are present in the hollow fiber membrane, or when only one dense layer is present at an equal distance from either surface, the observation method of “surface 1” is applied to both the inner surface and the outer surface.

The pore size formed on the surface of the hollow fiber membrane, that is, the pore size of the surface 1 or the surface 2 can be measured from an image of the surface observed with a scanning electron microscope (SEM). In the SEM observation, the area of all pores in an area of 20 μm×20 μm is measured for pores whose surface (surface 1) on the side close to the dense layer can be confirmed at a magnitude of 3,000. When the total number of the pores measured is less than 50, the measurement in the area of 20 μm×20 μm is repeated until the total pore numbers reached at least 50 to add to the data.

For the surface (surface 2) farther from the dense layer, the area of all pores in an area of 40 μm×40 μm is measured for pores whose surface (surface 2) on the side farther from the dense layer can be confirmed at a magnitude of 1,500. When the total number of the pores measured is less than 50, the measurement in the area of 40 μm×40 μm is repeated until the total pore numbers reached at least 50 to add to the data.

The hollow fiber membrane preferably has a dense layer. By having the dense layer, there are a dense region (dense layer) contributing to the separation of the substance to be removed and a coarse region (coarse layer) having a low water permeation resistance and a large pore size so that both the separation performance and the water permeability are easily achieved. The dense layer may be present at any position in the cross section of the separation membrane. For example, the dense layer may exist at a position close to the inner surface of the hollow fiber membrane, or may exist at a position close to the outer surface, or at a central portion of the cross section. In addition, the dense layer may be continuously present from the inner surface or the outer surface, or a plurality of dense layers may be present as described above. Although not particularly limited, when a polysulfone-based polymer is used as the material of the hollow fiber membrane, the dense layer is provided on the outer surface side from the viewpoint of ease of membrane structure control.

In the hollow fiber membrane, the thickness of the dense layer in the cross section of the hollow fiber membrane is preferably 1.0 μm or more and 10.0 μm or less. When the thickness of the dense layer is within the above range, it is possible to achieve both high permeability and a high separation function, and it is possible to suppress an increase in filtration pressure even when the treatment amount of the liquid to be treated is increased. From the viewpoint of reducing the permeation resistance of the substance, the thickness of the dense layer is preferably 7.0 μm or less, more preferably 6.5 μm or less, still more preferably 6.0 μm or less, and particularly preferably 5.5 μm or less. On the other hand, since the dense layer is a layer having an effect of improving the separation function of the hollow fiber membrane, the dense layer preferably has a thickness of a certain degree or more. That is, the thickness of the dense layer is more preferably 1.5 μm or more, still more preferably 2.0 μm or more, and particularly preferably 2.5 μm or more.

The thickness of the dense layer can be determined by observing a cross section of the hollow fiber membrane, that is, a cross section perpendicular to the axial direction at a magnification of 2,000 using a scanning electron microscope (SEM), and analyzing a photographed image with image processing software. Specifically, the captured image is binarized by determining a threshold value so that a structure part has high brightness and the other portions have low brightness. In a straight line drawn perpendicularly to a tangent line of the inner surface and/or the outer surface of the hollow fiber membrane, a region where a low brightness part having an area of 0.2 μm² or more is not observed, that is, a region where a pore having a pore size of 0.5 μm or more is not observed when assuming that the pore shape is a perfect circle is specified as a dense layer, and the average value of the thickness of the dense layer in the cross section is obtained. More specifically, the thickness is measured by the method described in “(10) Measurement of thickness of dense layer” in Examples described later. However, when the structure part and the other portions cannot be separated due to a difference in contrast in the image, the image analysis may be performed by filling portions other than the structure part with black. Furthermore, as a method of erasing noise, a noise part may be painted white.

Examples of the method of setting the thickness of the dense layer within the above range include a method of controlling the thickness by the composition of a spinning dope solution or the spinneret temperature at the time of spinning. More specifically, there is a method of increasing the viscosity by increasing the content of the main component (for example, polysulfone) constituting the hollow fiber membrane in the spinning dope or increasing the added amount of the high molecular weight polymer (for example, polyvinylpyrrolidone).

The higher the open pore ratio of the surface of the hollow fiber membrane, the less the permeation resistance and the higher the water permeability. Therefore, the open pore ratio of the surface (surface 1) closer to the dense layer is preferably 20% or more and more preferably 25% or more. The open pore ratio of the surface (surface 2) farther from the dense layer is preferably 20% or more. On the other hand, when the open pore ratio is excessively high, the strength of the separation membrane decreases, and therefore the open pore ratio of the surface closer to the dense layer is preferably 50% or less and more preferably 40% or less. The open pore ratio of the surface farther from the dense layer is preferably 40% or less and more preferably 35% or less.

The open pore ratio of the surface of the hollow fiber membrane is measured in the same manner as the pore size of the surface described above, and calculated using the following equation, and rounded off to the two decimal place to calculate the open pore ratio.

$$Open pore ratio ( \% ) = S / A \times 100$$

A: Area (μm²) of measurement range, and S: Sum of areas (μm²) of measured pores.

In the hollow fiber membrane, the inner diameter of the hollow fiber membrane is preferably 150 μm or more and 500 μm or less. From the viewpoint of increasing a certain effective hollow fiber membrane area without increasing the number of hollow fiber membranes and reducing the pressure loss during use, the inner diameter of the hollow fiber membrane is more preferably 200 μm or more, still more preferably 250 μm or more, and particularly preferably 300 μm or more. On the other hand, when the inner diameter is excessively increased, the module size increases and the capacity increases, and thus the inner diameter of the hollow fiber membrane is more preferably 450 μm or less, still more preferably 400 μm or less, and particularly preferably 350 μm or less.

Examples of the method of setting the inner diameter of the hollow fiber membrane within the above range include a method of adjusting the ejecting amount of the core solution during spinning.

The membrane thickness of the hollow fiber membrane is preferably 20 μm or more and 100 μm or less. From the viewpoint of the strength of the hollow fiber membrane, the membrane thickness of the hollow fiber membrane is more preferably 30 μm or more, still more preferably 40 μm or more, and particularly preferably 50 μm or more. On the other hand, the membrane thickness of the hollow fiber membrane is more preferably 90 μm or less, still more preferably 80 μm or less, and particularly preferably 70 μm or less because the membrane thickness part becomes permeation resistance of the substance.

Examples of the method of setting the membrane thickness of the hollow fiber membrane within the above range include a method of adjusting the ejecting amount of the spinning dope solution during spinning.

The hollow fiber membrane preferably has a three-dimensional network structure. The three-dimensional network structure refers to a structure in which a solid content spreads three-dimensionally in a network shape. The three-dimensional network structure has pores partitioned into solid components forming a network.

In a separation membrane used for blood treatment applications, particularly plasma separation applications, high water permeability is required to suppress hemolysis and activation of platelets and the like due to a load on hemocyte components. When the water permeability of the separation membrane is high, the pressure required at the time of treatment of blood can be reduced, the load on the blood can be reduced, and the treatment can be performed in a short time. From such a viewpoint, the water permeability of the separation membrane is preferably 16,000 mL/h/mmHg/m² or more and more preferably 17,000 mL/h/mmHg/m² or more. On the other hand, even when the water permeability is excessively high, stimulation to hemocytes becomes strong, and thus the water permeability is preferably 30,000 mL/h/mmHg/m² or less and more preferably 25,000 mL/h/mmHg/m² or less. The water permeability is calculated by the method described in Examples.

The hollow fiber membrane preferably has an asymmetric structure. As the separation membrane having particle permeability and water permeability described above, it is preferable to have an asymmetric structure in which the pore size on the other surface side is larger or smaller than the pore size on one surface side. That is, the asymmetric structure means that the pore sizes are different between the inner surface and the outer surface of the membrane.

A hollow fiber membrane is used as the separation membrane. The hollow fiber membrane can increase the membrane area effective for filtration even with a module having a small volume as compared with a flat membrane, that is, the module can be downsized. From the viewpoint of increasing the effective membrane area, the cross-sectional shape of the hollow fiber membrane may be a cross shape, a star shape or the like.

In the hollow fiber membrane, from the viewpoint of strength, it is preferable that no macrovoid, which is a hollow region where a real part of the membrane is missing in an elliptical shape or a drop shape, is observed in the cross section of the hollow fiber membrane.

A second aspect of the hollow fiber membrane is a hollow fiber membrane, which has permeability of a vesicle detectable by antibodies of phosphatidylserine and CD9 is 50% or more and 100% or less, and is used to separate the vesicle from a biological component. The vesicle detectable by the antibodies of phosphatidylserine and CD9 refers to a vesicle having phosphatidylserine and CD9 as surface markers.

Examples of the method of setting the permeability of the vesicle that can be detected by the antibodies of phosphatidylserine and CD9 within the above range include a method of adjusting the particle permeability of the hollow fiber membrane and the average pore size of the inner surface to those described above, a method of adjusting the pore size distribution in the separation membrane, and a method of adjusting a connecting structure of pores in the separation membrane. This is because the vesicles that can be detected by the antibodies of phosphatidylserine and CD9 are a population of exosomes and microvesicles that are distributed within a relatively narrow range of diameters and rigidity. A separation membrane in which the particle permeability and the average pore size of the inner surface are adjusted to those described above is preferable because it is excellent in the permeability of the entire vesicles. In addition, by adjusting the pore size distribution in the hollow fiber membrane, a substance that permeates and a substance that does not permeate can be clearly separated, blockage by components (for example, hemocytes, proteins having a large size and the like) other than the vesicle inevitably contained in the liquid to be treated hardly occurs, and the vesicle that can be detected by the antibodies of phosphatidylserine and CD9 can be effectively permeated. As the connecting structure of the pores in the separation membrane, by designing the phase separation conditions so that there is no stenosis in the permeation path of the vesicle, the vesicle that can be detected by the antibodies of phosphatidylserine and CD9 can be efficiently permeated.

The hollow fiber membrane is preferably used for blood purification. As described above, the blood purification is a treatment method by correcting a body fluid and removing a biotoxin. Blood purification includes hemodialysis, plasma exchange therapy, adsorption therapy and the like.

In the hollow fiber membrane, for example, when the hollow fiber membrane is used for blood purification, the average depth of pores in the inner surface is preferably 0.15 μm or more and 7.00 μm or less from the viewpoint of preventing activation of platelets. Generally, when the pore size of the inner surface of the hollow fiber membrane is increased, the depth of the pore of the inner surface, that is, the roughness of the surface tends to increase so that the stimulation to hemocytes increases, and platelets are easily activated. Therefore, from the viewpoint of suppressing platelet activation, the average depth of pores in the inner surface is more preferably 6.00 μm or less, still more preferably 5.00 μm or less, and particularly preferably 4.00 μm or less. On the other hand, from the viewpoint of improving the vesicle permeability, the average depth of the pores of the inner surface is more preferably 0.50 μm or more, still more preferably 1.00 μm or more, and particularly preferably 1.50 μm or more. Examples of the method of controlling the average depth of the pores in the inner surface include a method of adjusting the coagulation value and viscosity of the injection liquid.

### Hydrophilic Polymer

The hollow fiber membrane preferably contains a hydrophilic polymer. As a method in which the hollow fiber membrane contains a hydrophilic polymer, inclusion in a closed void structure, adhesion (physical adsorption), chemical immobilization and the like can be considered, but the method is preferably adhesion or chemical immobilization. By supporting the hydrophilic polymer on the surface of the separation membrane, adsorption of proteins in blood and clogging due to adsorption can be suppressed, protein permeability can be improved, and coagulation of blood can be suppressed.

The hydrophilic polymer is a water-soluble polymer or a polymer that is water-insoluble, but interacts with water molecules through an electrostatic interaction or a hydrogen bond. The water-soluble polymer refers to a polymer that is dissolved in pure water at 25° C. at a ratio of 1,000 ppm (0.1 g/mL) or more. Specific examples of the water-soluble polymer include polyalkylene glycols such as polyethylene glycol or polypropylene glycol, and ionic hydrophilic polymers such as polyvinyl alcohol, polyvinyl pyrrolidone (hereinafter, “PVP”), dextran sulfate, polyacrylic acid, polyethyleneimine, or polyallylamine. Examples of the hydrophilic polymer that interacts with water molecules by electrostatic interaction or hydrogen bonding even though it is water-insoluble include nonionic hydrophilic polymers such as polyvinyl acetate, polyvinyl caprolactam, hydroxyethyl methacrylate, and methyl methacrylate.

The hydrophilic polymer may be copolymerized with another monomer.

In the hollow fiber membrane, when containing a hydrophilic polymer, the content of the hydrophilic polymer is preferably 5.0 mass % or more and 15.0 mass % or less. The content of the hydrophilic polymer is more preferably 7.0 mass % or more, still more preferably 9.0 mass % or more, even more preferably 11.0 mass % or more, and particularly preferably 12.0 mass % or more from the viewpoint of improving the wettability of the hollow fiber membrane and improving the water permeability and permeability as the hydrophilic polymer is contained in the hollow fiber membrane. On the other hand, as the content of the hydrophilic polymer in the hollow fiber membrane decreases, elution during use can be suppressed, and a change in performance of the hollow fiber membrane can be suppressed. Therefore, the content of the hydrophilic polymer is more preferably 14.5 mass % or less and particularly preferably 14.0 mass % or less.

For the content of the hydrophilic polymer in the hollow fiber membrane, it is necessary to select a measurement method depending on the type of the polymer, but in this disclosure, a value obtained by elemental analysis is used.

A part of the hydrophilic polymer is preferably insolubilized in the hollow fiber membrane. That is, it is preferable that the hollow fiber membrane contains an insoluble component that is insoluble in a good solvent with respect to a main component of the hollow fiber membrane, the insoluble component contains the same hydrophilic unit as the hydrophilic polymer, and when dissolved in N,N-dimethylacetamide (hereinafter, “DMAc”), a ratio of a mass of the insoluble component to a dry mass of the hollow fiber membrane is 1 mass % or more and 45 mass % or less.

From the viewpoint of suppressing elution of the hydrophilic polymer from the hollow fiber membrane, the proportion of the insoluble component is more preferably 3 mass % or more, still more preferably 5 mass % or more, particularly preferably 6 mass % or more, and most preferably 7 mass % or more. On the other hand, the more the degree of crosslinking, the higher the effect of suppressing the adhesion of hemocyte components, and thus the degree of crosslinking is more preferably 35 mass % or less, still more preferably 25 mass % or less, particularly preferably 20 mass % or less, and most preferably 15 mass % or less.

The hydrophilic polymer may be appropriately selected depending on the material of the hollow fiber membrane and the affinity with the solvent. In a polysulfone-based polymer, the preferred is use of polyvinylpyrrolidone (PVP) in view of the high compatibility although not particularly limited.

In a hollow fiber membrane used for blood treatment, the hydrophilic polymer on a surface (inner surface is preferable) in contact with blood is important. Insufficient amount of the hydrophilic polymer on such surface will lead to an insufficient blood compatibility, and aggregation of platelets, that is, an increased risk of blood coagulation. Therefore, the hydrophilic polymer on the surface in contact with blood is preferably 40 mass % or more, more preferably 45 mass %, and still more preferably 50 mass % or more. On the other hand, when the hydrophilic polymer is large, the hydrophilic polymer eluted into blood increases, and there is a possibility that the performance of the hollow fiber membrane is changed during use due to the elution of the hydrophilic polymer, and the hollow fiber membrane is difficult to handle. Therefore, the hydrophilic polymer on the surface is preferably 70 mass % or less, and more preferably 60 mass % or less.

The hydrophilic polymer on the surface of the hollow fiber membrane can be measured by means of X-ray photoelectron spectroscopy (XPS). The measurement is performed at a measurement angle of 90°. At the measurement angle of 90°, an area from the surface to the depth of about 10 nm are detected. The value used is the average of measurements at 3 different sites. For example, when the hydrophobic polymer is a polysulfone and the hydrophilic polymer is a PVP, content of the PVP on the surface is calculated from the measurements of nitrogen content (d (atom %)) and the sulfur content (e (atom %)) by the following equation: PVP content (f)=100×(d×111)/(d×111+e×442).

The hollow fiber membrane preferably contains a plurality of kinds of hydrophilic polymers. One of them is preferably a polymer (so-called “biocompatible polymer”) having an effect of suppressing adhesion of a biological component contained in blood, plasma, urine or the like, particularly a protein. That is, the hollow fiber membrane is preferably a hollow fiber membrane containing a hydrophilic polymer and a biocompatible polymer.

The biocompatible polymer refers to a polymer having the number of platelets attached to the surface of 50 platelets/103 μm² or less in a platelet adhesion test. The test of attachment of platelets can be performed by the method described in “(12) Test of attachment of platelets” in Examples described later using a resin made of a biocompatible polymer, a polymer film obtained by modifying a surface of a biocompatible polymer or the like.

The hydrophilic polymer (biocompatible polymer) exhibiting biocompatibility is not particularly limited, and examples thereof include hydrophilic polymers such as polyethylene glycol, polyethyleneimine, polyvinyl alcohol, and PVP and derivatives thereof, 2-methacryloyloxyethyl phosphorylcholine (MPC) and derivatives thereof, 2-methoxyethyl acrylate (PMEA) and derivatives thereof, and polymers containing an ester group described later. Among them, a biocompatible polymer containing an ester group is particularly preferable from the viewpoint of chemical stability and little influence on pH. Furthermore, among the biocompatible polymers containing an ester group, a polymer containing a monocarboxylic acid vinyl ester unit is particularly preferable. That is, in the hollow fiber membrane, it is particularly preferable that at least one of the hydrophilic polymers is a hydrophilic polymer containing a monocarboxylic acid vinyl ester unit as a repeating unit.

Regarding the polymer containing a monocarboxylic acid vinyl ester unit, the monocarboxylic acid means a compound including one carboxy group and a hydrocarbon group bonded to a carbon atom of the carboxy group, that is, a compound represented by “R—COOH” (R is a hydrocarbon group). The hydrocarbon group R may be either an aliphatic hydrocarbon group or an aromatic hydrocarbon group, but from the viewpoint of ease of synthesis and the like, an aliphatic hydrocarbon group is preferable, and a saturated aliphatic hydrocarbon group is more preferable. Examples of the saturated aliphatic hydrocarbon group include those having a linear structure such as an ethyl group, a n-propyl group, a n-butyl group, a n-pentyl group, and a n-hexyl group, those having a branched structure such as an isopropyl group and a tertiary butyl group, and those having a cyclic structure such as a cyclopropyl group and a cyclobutyl group. Furthermore, an ether bond, an ester bond or the like may be included in the aliphatic chain. Among them, from the viewpoint of the production cost of the carboxylic acid, the saturated aliphatic hydrocarbon group preferably has a linear structure or a branched structure, and more preferably has a linear structure.

Examples of the monocarboxylic acid in which the hydrocarbon group R is an aromatic hydrocarbon group include benzoic acid and a derivative thereof. Examples of the monocarboxylic acid in which the hydrocarbon group R is a saturated aliphatic hydrocarbon group include acetic acid, propanoic acid, and butyric acid.

In the hydrocarbon group R, at least a part of hydrogen atoms may be optionally substituted. In this instance, the substituent is preferably a nonpolar group or a cationic functional group, since protein denaturation due to contact and accompanying protein adsorption to the hollow fiber membrane surface are less likely to occur.

The hydrocarbon group R preferably has a small number of carbon atoms from the viewpoint of lowering the hydrophobicity of the monocarboxylic acid to reduce hydrophobic interaction with a protein and thus prevent attachment. Therefore, when R is an aliphatic hydrocarbon group or an aromatic hydrocarbon group, the number of carbon atoms is preferably 20 or less, more preferably 9 or less, and still more preferably 5 or less. On the other hand, when R is an aliphatic hydrocarbon group or an aromatic hydrocarbon group, the number of carbon atoms is 1 or more, but is preferably 2 or more from the viewpoint of improving the mobility of the monocarboxylic acid and further suppressing adhesion of proteins. When R is a saturated aliphatic hydrocarbon group, the compound having one carbon atom is acetic acid, and the compound having two carbon atoms is propanoic acid.

The “unit” refers to a repeating unit in a homopolymer or copolymer obtained by polymerizing a monomer, and the “carboxylic acid vinyl ester unit” means a repeating unit obtained by polymerizing a carboxylic acid vinyl ester monomer, that is, a repeating unit represented by “—CH(OCO—R)—CH₂—” (R is a hydrocarbon group). R is the same as described for the monocarboxylic acid above, and preferred examples and the like also follow the above description.

Furthermore, from the viewpoint of a balance between hydrophilicity and hydrophobicity, the “carboxylic acid vinyl ester unit” is preferably a “monocarboxylic acid vinyl ester unit” obtained by polymerizing a monocarboxylic acid vinyl ester monomer.

Specific examples of the monocarboxylic acid vinyl ester unit in which the hydrocarbon group R is a saturated aliphatic hydrocarbon group include a vinyl propanoate unit, a vinyl pivalate unit, a vinyl decanoate unit, and a vinyl methoxyacetate unit. Preferred examples thereof include a vinyl acetate unit (R: CH₃), a vinyl propanoate unit (R: CH₂CH₃), a vinyl butyrate unit (R: CH₂CH₂CH₃), a vinyl pentanoate unit (R: CH₂CH₂CH₂CH₃), a vinyl pivalate unit (R: C(CH₃)₃), and a vinyl hexanoate unit (R: CH₂CH₂CH₂CH₂CH₃) since the hydrophobicity is preferably not excessively strong. Specific examples of the monocarboxylic acid vinyl ester unit in which R is an aromatic group include a vinyl benzoate unit and its substituted products.

As a method of confirming that the biocompatible polymer is supported on the hollow fiber membrane, when the biocompatible polymer is a monocarboxylic acid vinyl ester unit is shown as an example. When another polymer is used as the biocompatible polymer, an ion signal derived from a molecular structure peculiar to the used biocompatible polymer is measured by appropriately combining a time-of-flight secondary ion mass spectrometer (hereinafter, sometimes referred to as “TOF-SIMS”) and other measurement methods.

The fact that the polymer containing a monocarboxylic acid vinyl ester unit is supported on the hollow fiber membrane can be confirmed by combining measurement by a TOF-SIMS apparatus and measurement by X-ray photoelectron spectroscopy (XPS). Specifically, first, a peak derived from a carboxylic acid ion of the saturated aliphatic monocarboxylic acid ester is detected by measurement using the TOF-SIMS apparatus, and therefore the structure of the monocarboxylic acid is revealed by analyzing the mass (m/z).

In the measurement by the TOF-SIMS apparatus, pulsed ions (primary ions) are applied to a sample surface placed in ultrahigh vacuum, and ions (secondary ions) released from the sample surface are given certain kinetic energy, and guide to a time-of-flight mass analyzer. Each of the secondary ions accelerated with the same energy passes through the analyzer at a speed corresponding to the mass, and since the distance to the detector is constant, the time taken to reach the detector (flight time) is a function of the mass, and the distribution of the flight time is accurately measured to obtain a secondary ion mass distribution, that is, a mass spectrum. For example, when a secondary positive ion is detected using Bi³⁺⁺ as primary ion species, the peak at m/z=43.01 corresponds to C₂H₃O^{′}, i.e. acetic acid (the number of carbon atoms in an aliphatic chain is 1). In addition, the peak at m/z=57.03 corresponds to C₃H₅O^{′}, that is, propanoic acid (the number of carbon atoms in an aliphatic chain is 2).

Conditions for measurement of the ion signal by the TOF-SIMS device are as follows. The measurement region has a size of 100 μm×100 μm, the primary ion acceleration voltage is 30 kV, and the pulse width is 7.8 ns. The detection depth in this analysis method is several nanometers or less. When the strength of the ion signal derived from the carboxylic acid is 0.1% or less with respect to the total secondary ion strength, the value of the carboxylic acid ion strength is judged ascribable to noise, and it is determined that there is no carboxylic acid ion.

Further, when performing the XPS measurement, the peak of carbon derived from an ester group (COO) appears at +4.0 to 4.2 eV from the main peak of CHx or C—C (around 285 eV), and therefore it is understood that the carboxylic acid forms an ester bond. A value measured at 90° as a measurement angle in XPS is used. When measurement is performed at a measurement angle of 90°, a region with a depth of about 10 nm from the surface is detected. The peak of carbon derived from an ester group (COO) can be determined by peak-dividing a peak appearing at +4.0 to 4.2 eV from the main peak derived from CH or C—C in C1s. More specifically, the peak of C1s is composed of five components: a component derived mainly from CHx, C—C, C═C or C—S, a component derived mainly from C—O or C—N, a component derived from a π-π* satellite, a component derived from C—O, and a component derived from COO. By performing peak division into the above five components and calculating the ratio of the peak area derived from the ester group to the total peak area derived from the carbon, the amount of carbon derived from the ester group (number of atoms %) can be determined. When the ratio of the area of a peak derived from an ester group to the total area of peaks derived from carbon is 0.4% or less, the value of the area of the peak is judged as ascribable to noise, and it is determined that there is no ester group.

In the hollow fiber membrane for blood treatment applications, particularly in plasma separation applications, proteins in plasma pass through the interior of the separation membrane and are separated from hemocyte components. Therefore, from the viewpoint of suppressing adhesion of proteins, the biocompatible polymer is preferably supported on the entire separation membrane. From this viewpoint, the amount of carbon atoms derived from the ester group (atom number %) is preferably 1% or more and preferably 10% or less on both the blood-contacting surface and the opposite surface.

The fact that the biocompatible polymer is supported on the entire separation membrane can be confirmed by performing the TOF-SIMS measurement described above on the cross section of the separation membrane.

The weight average molecular weight of the biocompatible polymer is preferably 1,000 or more and more preferably 5,000 or more from the viewpoint of sufficiently suppressing adhesion of proteins and further improving protein permeability. On the other hand, the weight average molecular weight of the biocompatible polymer is preferably 1,000,000 or less, more preferably 500,000 or less, and still more preferably 100,000 or less from the viewpoint of introduction efficiency into the separation membrane. The weight average molecular weight of the biocompatible polymer can be measured by gel permeation chromatography (GPC).

In particular, when the biocompatible polymer is supported on the entire separation membrane, the molecular weight is preferably smaller than the pore size of the separation membrane and easily permeates the separation membrane.

The biocompatible polymer, particularly the polymer containing a monocarboxylic acid vinyl ester unit is preferably a copolymer (hereinafter, it may be simply referred to as a “copolymer”) made of a hydrophilic unit and a hydrophobic unit, and the hydrophobic unit more preferably contains a monocarboxylic acid vinyl ester unit. When the surface of the separation membrane is coated with a hydrophilic polymer such as polyethylene glycol or polyvinyl alcohol, the effect of suppressing adhesion of proteins and the like may be insufficient. It is considered that this is because when the hydrophilicity of the surface of the separation membrane is excessively strong, the structure of the protein becomes unstable, and thus the attachment of the protein cannot be sufficiently suppressed. In particular, in recent years, water around a polymer has attracted attention. A polymer having high hydrophilicity has a strong interaction with water, and the mobility of water around the polymer decreases. On the other hand, it is considered that the structure of a protein is stabilized by water called adsorbed water. The adsorbed water is a generic term for water (refer to, for example, Polymer Society of Japan, 2014 Vol. 63, August No. 542) in a state also referred to as “non-freezing bound water” or “intermediate water”. Therefore, it is considered that when the state of adsorbed water of the protein is similar to that of the adsorbed water around the polymer, the structure of the protein is not destabilized, and adhesion of the protein to the surface of the separation membrane can be suppressed. It is considered that the copolymer including the hydrophilic unit and the hydrophobic unit can control the state of adsorbed water around the polymer by selecting the hydrophilic group, the hydrophobic group, and the copolymerization ratio to be used, and can further improve the effect of suppressing protein adhesion. The hydrophilic unit refers to a unit in which a polymer having a weight average molecular weight of 10,000 to 1,000,000 of a monomer alone constituting the unit is soluble in water. The term “soluble” refers to having a solubility of more than 0.1 g in 100 g of water at 20° C.

The monomer constituting the hydrophilic unit is more preferably a monomer having a solubility exceeding 10 g. Examples of such a monomer include a vinyl alcohol monomer, an acryloylmorpholine monomer, a vinylpyridine-based monomer, a vinylimidazole-based monomer, and a vinylpyrrolidone monomer. Two or more of these may be used. In particular, monomers having an amide bond, an ether bond or an ester bond are preferable because they do not have excessively high hydrophilicity, and are more easily balanced with a hydrophobic monomer as compared to monomers having a carboxy group or a sulfonic acid group. Particularly, vinylacetamide monomers having an amide bond, vinylpyrrolidone monomers and vinylcaprolactam monomers are more preferable. Among them, vinylpyrrolidone monomers are still more preferable because the polymer has low toxicity. Therefore, the biocompatible polymer further contains a vinylpyrrolidone unit as the hydrophilic unit.

Examples of the monomer constituting the hydrophobic unit include at least monocarboxylic acid vinyl ester, and other monomers include acrylic acid ester, methacrylic acid ester, and vinyl-ε-caprolactam.

From the viewpoint of further suppressing protein adhesion, the mole fraction of the hydrophobic unit in the whole copolymer made of the hydrophilic unit and the hydrophobic unit is preferably 10% or more and 90% or less, more preferably 20% or more and 80% or less, and still more preferably 30% or more and 70% or less. At this time, the hydrophobic unit may include only a monocarboxylic acid vinyl ester unit or may include another hydrophobic unit. By setting the mole fraction of the hydrophobic unit to 90% or less, an increase in hydrophobicity of the entire copolymer can be suppressed, and adhesion of proteins can be further suppressed. On the other hand, by setting the mole fraction of the hydrophobic unit to 10% or more, it is possible to suppress an increase in the hydrophilicity of the entire copolymer, avoid structural destabilization and denaturation of proteins, and further suppress adhesion. The mole fraction can be calculated, for example, from a peak area obtained by nuclear magnetic resonance (NMR) measurement. If the mole fraction cannot be calculated by the NMR measurement for the reasons such as overlap of the peaks, the mole fraction may be calculated by elemental analysis.

The biocompatible polymer is particularly preferably a copolymer including a monocarboxylic acid vinyl ester unit and a vinylpyrrolidone unit. In this instance, the mole ratio of the vinylpyrrolidone unit to the monocarboxylic acid vinyl ester unit is preferably 30:70 to 90:10, more preferably 40:60 to 80:20, and still more preferably 50:50 to 70:30.

Examples of the sequence of units in the above copolymer include a block copolymer, an alternating copolymer, and a random copolymer. Among them, an alternating copolymer or random copolymer is preferable because the copolymer as a whole has a small distribution of hydrophilicity/hydrophobicity. Among them, a random copolymer is more preferable from the viewpoint of ease of synthesis.

Although not essential, from the viewpoint of avoiding elution of the biocompatible polymer from the hollow fiber membrane during use for filtration, the biocompatible polymer is preferably immobilized on the separation membrane by chemical bonding. A method of immobilization will be described later.

The material constituting the main component of the hollow fiber membrane is preferably an amorphous polymer. An amorphous polymer is a polymer which does not experience crystallization, and which shows no exothermic peak by the crystallization in the measurement by differential scanning calorimeter.

Since the amorphous polymer is likely to cause structural deformation, it is easy to control the structure in a membrane thickness direction. It has been known that a hollow fiber membrane made of the amorphous polymer is prepared by inducing phase separation of a membrane formation dope solution that has been prepared by dissolving the amorphous polymer in a solvent by using heat or poor solvent and removing the solvent component. Since the amorphous polymer in the solvent is highly mobile, the amorphous polymer aggregates during the phase separation, and this enables increase in the concentration and formation of a dense structure. By using different phase separation speed in the membrane thickness direction, a membrane having an asymmetrical structure wherein pore size is different in the membrane thickness direction can be prepared.

Examples of the amorphous polymer used for the material of the hollow fiber membrane include acryl polymers, vinyl acetate polymers, and polysulfone-based polymers. Of these, the preferred is used of a polysulfone-based polymer in view of easy control of the pore size. That is, the hollow fiber membrane preferably contains a polysulfone-based polymer. Furthermore, the hollow fiber membrane is more preferably a hollow fiber membrane containing a polysulfone-based polymer as a main component. The main component means the largest component on a mass basis in the hollow fiber membrane.

The term “polysulfone-based polymer” is a polymer having an aromatic ring, sulfonyl group, and ether group in the backbone. Exemplary non-limiting preferable polysulfone-based polymers include those represented by following chemical formulae (1) and (2). For example, a molecule, a polymer or the like may be grafted by introducing a sulfonic acid group into a part of the aromatic ring. In the formula, n is, for example, an integer of 50 to 80.

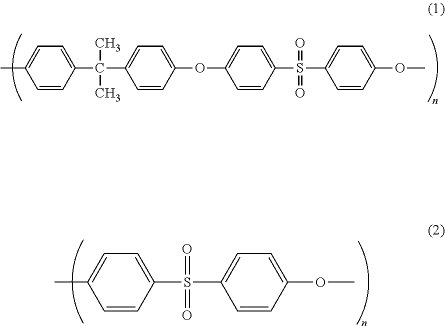

Examples of the polysulfone include “Udel” (registered trademark) polysulfone P-1700 and P-3500 (manufactured by Solvay), “Ultrason” (registered trademark) S3010 and S6010 (manufactured by BASF). Although the polysulfone is preferably a polymer solely comprising the constitutional repeating unit represented by the formula (1) and/or (2), the polysulfone may be in the form of a copolymer with other monomer to the extent not adversely affecting the merits. Content of such other monomer is preferably 10 mass % or less although such content is not particularly limited.

When a hydrophilic polymer is added to the membrane formation dope solution, the resulting hollow fiber membrane will contain the hydrophilic polymer, and the thus improved water wettability results in the improved water permeability. In addition, the viscosity of the membrane formation dope solution can be adjusted, that is, the pore size can be adjusted.

Examples of the method of supporting the biocompatible polymer on the hollow fiber membrane include a method in which the biocompatible polymer is added to a dope solution or a core solution during membrane formation, and a method in which the biocompatible polymer solution is brought into contact with the surface after membrane formation. Among them, the method in which the biocompatible polymer solution is brought into contact with the surface after membrane formation is preferable from the viewpoint of not affecting membrane formation conditions. As such a method, any method such as a method of immersing the hollow fiber membrane in a biocompatible polymer solution, a method of passing the solution, or a method of spraying the solution with a spray may be used. Among them, a method of allowing the biocompatible polymer solution to pass through the hollow fiber membrane is preferable because the biocompatible polymer can be applied to the inside of the hollow fiber membrane.

When the biocompatible polymer solution is allowed to pass through the separation membrane, from the viewpoint of more efficiently introducing the biocompatible polymer, the concentration of the biocompatible polymer in the biocompatible polymer solution is preferably 10 ppm or more, more preferably 100 ppm or more, and still more preferably 300 ppm or more. On the other hand, from the viewpoint of suppressing elution from the module, the concentration of the coating polymer in the aqueous solution is preferably 100,000 ppm or less and more preferably 10,000 ppm or less.

The solvent used for preparing the biocompatible polymer solution is preferably water. However, when the biocompatible polymer is not dissolved in water at a predetermined concentration, it may be dissolved in a mixed solvent of water and an organic solvent in which the hollow fiber membrane is insoluble, or an organic solvent which is compatible with water and in which the hollow fiber membrane is insoluble. Examples of the organic solvent that can be used in the organic solvent or the mixed solvent include, but are not limited to, alcohol-based solvents such as methanol, ethanol, and propanol.

The direction in which the biocompatible polymer solution is allowed to pass through the separation membrane may be either the coarse layer side from the dense layer side of the hollow fiber membrane or the coarse layer side from the dense layer side, but it is preferable to allow the biocompatible polymer solution to pass through the hollow fiber membrane from the coarse layer side to the dense layer side from the viewpoint of efficiently imparting the biocompatible polymer to the inside of the hollow fiber membrane. When the size of the biocompatible polymer to be used is larger than the pore size of the dense layer, if the biocompatible polymer is passed from the dense layer side, the biocompatible polymer does not pass through the pores and is concentrated on the surface of the dense layer so that it may be difficult to support the biocompatible polymer on the coarse layer.

As described above, the biocompatible polymer is preferably immobilized on the separation membrane by chemical bonding. The method of immobilization by chemical bonding is not particularly limited, and examples thereof include a method in which a coating polymer is brought into contact with the separation membrane and then irradiated with radiation, and a method in which a reactive group such as an amino group or a carboxy group is introduced onto the surface of the coating polymer and the separation membrane to be immobilized and condensed. Method of producing hollow fiber membrane

Examples of the method of producing the hollow fiber membrane include a method of ejecting an injection liquid (core solution) or an injection gas from the cylindrical tube in the interior a double tube spinneret and ejecting the membrane formation dope solution from the slit on the outer side. The structure of the inner surface of the hollow fiber membrane can be controlled by adjusting the concentration and temperature of the poor solvent of the core solution, or by adding the additive. On the other hand, the structure of the outer surface side can be controlled by the atmospheric conditions of the dry section and the composition of the coagulation bath until the liquid is ejected from the spinneret and enters the coagulation bath. It is preferable to form a hollow fiber membrane having an asymmetric structure in which the pore size on the inner surface side is large because the structure on the inner surface of the hollow fiber membrane is easily controlled as compared with the outer surface.

As the hollow fiber membrane, an example of a method of producing a hollow fiber membrane containing the above-described polysulfone-based polymer as a main component will be described. As a method of forming a hollow fiber membrane, a phase separation method is preferred. As the phase separation method, a method of inducing phase separation with a poor solvent (non-solvent induced separation method, NIPS), a method of inducing phase separation by cooling a membrane formation dope solution at a high temperature using a solvent having relatively low solubility (Thermally Induced Phase Separation, TIPS) and the like can be used, and among them, membrane formation by the method of inducing phase separation with a poor solvent is particularly preferable.

For example, there is a membrane formation process in which a membrane formation dope solution containing a polysulfone-based polymer is simultaneously ejected from an outer cylinder and a core solution is simultaneously ejected from an inner cylinder using an orifice-type double cylindrical spinneret, passed through a dry section, then immersed in a coagulation bath containing a coagulation solution to be coagulated, and further washed with warm water.

The concentration of the polysulfone-based polymer contained in the membrane formation dope solution is preferably 10 to 25 mass % and more preferably 10 to 20 mass %. The concentration of the polysulfone-based polymer greatly affects the open pore ratio on the surface of the hollow fiber membrane. When the polysulfone-based polymer concentration is 25 mass % or less, the cohesive force between the polysulfone-based polymers is weakened in the membrane formation process, the pressure in the membrane formation process is less likely to increase, and the open pore ratio on the surface of the separation membrane is likely to be improved. On the other hand, when the concentration of the polysulfone-based polymer is 10 mass % or more, although the open pore ratio is low, the strength of the hollow fiber membrane is improved so that fiber breakage hardly occurs.

By using a hydrophilic polymer having a relatively low molecular weight (weight average molecular weight of 1,000 to 200,000), the pore forming action is enhanced, the open pore ratio is improved, and the water permeability of the hollow fiber membrane can be improved, but the pore size is relatively small. On the other hand, when a hydrophilic polymer having a relatively high molecular weight (weight average molecular weight of 200,000 to 1,200,000) is used, the hydrophilic polymer has a long molecular chain and has a large interaction with the polysulfone-based polymer, and therefore easily remains in the hollow fiber membrane, contributing to improvement of hydrophilicity of the hollow fiber membrane. The pore size is also relatively large.

The hydrophilic polymer added to the membrane formation dope solution may be used alone, or two or more kinds thereof may be mixed, or hydrophilic polymers having different molecular weights may be blended.

When preparing a membrane formation dope solution, it is preferable to dissolve at a high temperature of 30° C. or higher to improve solubility. On the other hand, when the polymer is dissolved at 120° C. or lower, it is possible to suppress a change in composition due to modification of the polymer by heat or evaporation of the solvent. Accordingly, the dissolution temperature is preferably 30° C. or higher and 120° C. or lower. However, the optimal solution temperature may vary by the types of the polymer and the additives used.

The core solution in the membrane formation process refers to a liquid present in the hollow portion formed by the hollow fiber membrane in the production process and containing a good solvent for the polysulfone-based polymer, and examples thereof include DMAc, dimethylformamide, N-methylpyrrolidone, dimethylsulfoxide, glycerin, and a mixed solvent thereof. To increase the spinning stability, PVP, a copolymer containing vinylpyrrolidone, polyethylene glycol, polyvinyl alcohol, polyacrylic acid, polyethyleneimine or the like may be added to the core solution.

The composition of the core solution greatly affects the open pore ratio, average pore size, pore shape, and hydrophilic polymer content on the surface of the hollow fiber membrane. When the concentration of the good solvent contained in the core solution is increased, aggregation of polysulfone-based polymers can be alleviated, and a hollow fiber membrane having a high open pore ratio on the surface of the hollow fiber membrane and a large pore size can be obtained. When the hydrophilic polymer is added to the core solution, not only the hydrophilic polymer can be localized on the inner surface of the hollow fiber membrane, but also the hydrophilic polymer serves as a core to induce phase separation so that a hollow fiber membrane having a high hydrophilic polymer and a high open pore ratio on the inner surface of the hollow fiber membrane is obtained.

When the surface in contact with blood is the inner surface, the core solution temperature in the membrane formation process is preferably the same as or lower than the membrane formation dope solution temperature by 5° C. or higher, and more preferably by 10° C. or higher.

The dew point temperature of the dry section in the membrane formation process described above particularly greatly affects the outer surface of the hollow fiber membrane, but the phase separation reaction of the membrane formation dope solution can be controlled by controlling the dew point temperature and, for example, moisture can be supplied to the outer surface of the hollow fiber membrane to form a dense layer. When the surface in contact with blood is the inner surface, it is preferable that the dew point is adjusted to 20° C. to 40° C.

The length of the dry section in the membrane formation process described above determines the time from formation of pores on the surface of the hollow fiber membrane to coagulation, and is preferably 10 to 250 mm. When the length of the dry section is 10 mm or more, the average pore size on the surface of the hollow fiber membrane increases. On the other hand, when the length of the dry section is 250 mm or less, fiber swinging hardly occurs in the membrane formation process, and fiber breakage hardly occurs.

The coagulation solution in the membrane formation process refers to a poor solvent for the polysulfone-based polymer, and examples thereof include alcohol, water, and glycerin, and water is preferable.

The coagulation bath temperature in the membrane formation process is preferably 30° C. to 100° C., and more preferably 60° C. to 90° C. The coagulation bath temperature greatly affects the average pore size on the surface of the hollow fiber membrane and the water permeability of the hollow fiber membrane. When the coagulation bath temperature is 30° C. or higher, the pore size on the outer surface of the hollow fiber membrane increases, the permeable particle size increases, and the water permeability is improved. On the other hand, when the coagulation bath temperature is 100° C. or lower, the open pore ratio on the surface of the hollow fiber membrane is reduced, and the strength of the hollow fiber membrane is easily improved.

In addition to a coagulation solution such as water, a good solvent may be added to the coagulation bath in the membrane formation process at a ratio of 1 to 10 mass % with respect to the polysulfone-based polymer. When a good solvent is added, the coagulation solution diffusion rate in desolvation can be relaxed, and the structure on the outer surface side of the hollow fiber membrane can be made suitable. When the concentration of the good solvent is 1 mass % or more, it is easy to control the pore size on the outer surface and the thickness of the dense layer of the hollow fiber membrane. On the other hand, when the concentration of the good solvent is 10 mass % or less, desolvation of the hydrophilic polymer is appropriately promoted, and the strength of the hollow fiber membrane is easily improved.

The warm-water washing in the membrane formation process means that the hollow fiber membrane after being immersed in the coagulation bath is immersed in a warm water bath at 60° C. or higher for 1 minute or more. Excessive solvent and hydrophilic polymer remaining in the hollow fiber membrane are removed by warm-water washing. When the hydrophilic polymer is added to the core solution, to effectively remove the excessive hydrophilic polymer and improve the water permeability of the hollow fiber membrane, it is preferable to wind the hollow fiber membrane after warm-water washing, cut the hollow fiber membrane into a certain length, divide the hollow fiber membrane into small portions, and perform additional warm-water washing. As a specific example, it is preferable that a hollow fiber membrane bundle obtained by winding the hollow fiber membrane after warm-water washing, cutting the wound hollow fiber membrane into 400 mm, and dividing the cut hollow fiber membrane into small portions is wound with gauze, and the hollow fiber membrane bundle is additionally washed with warm water of 70° C. or higher for 1 to 5 hours. When the hollow fiber membrane is washed with warm water at 90° C. or higher, an excessive hydrophilic polymer which is not supported on and/or chemically immobilized on the hollow fiber membrane and a hydrophilic polymer filling pores of the hollow fiber surface membrane are eluted in the warm water so that those can be removed.

The hollow fiber membrane after the warm-water washing is in a wet state, but from the viewpoint of further stabilizing the water permeability of the hollow fiber membrane, a drying step is preferably performed. The temperature of the drying step is preferably 100° C. or higher from the viewpoint of evaporating moisture. The temperature of the drying step is preferably 180° C. or lower so as not to exceed the glass transition point of the polysulfone-based polymer.

In a separation membrane used for blood treatment, it is important to suppress elution of a hydrophilic polymer. To suppress elution of the hydrophilic polymer, the obtained hollow fiber membrane is preferably subjected to thermal crosslinking and radiation irradiation crosslinking.

In the thermal crosslinking for heating the obtained hollow fiber membranes, hydrophilic polymers present in the hollow fiber membranes are crosslinked. The temperature of thermal crosslinking is preferably 120° C. to 250° C., and more preferably 130° C. to 200° C., because a decomposition reaction can be made less likely to occur while the hydrophilic polymers are crosslinked. The time for thermal crosslinking is preferably 1 to 10 hours, and more preferably 3 to 8 hours.

In the radiation crosslinking in which the obtained hollow fiber membrane is irradiated with radiation, the hydrophilic polymer and the biocompatible polymer described above are crosslinked with the polysulfone-based polymer. The irradiation dose of the radiation crosslinking is preferably 5 to 75 kGy, and more preferably 10 to 50 kGy because the decomposition reaction can be made less likely to occur while the crosslinking reaction proceeds. As the radiation to be emitted, α rays, β rays, γ rays, X-rays, ultraviolet rays, electron beams or the like can be used. When a biocompatible molecule is supported, radiation is applied in a state in which a solution in which a biocompatible polymer is dissolved is brought into contact with a hollow fiber membrane in a hollow fiber membrane module described later, or in a state in which a biocompatible polymer is introduced into the surface and then the solution in the hollow fiber membrane module is removed, or in a state in which the hollow fiber membrane is dried. This method is preferable because sterilization of the hollow fiber membrane module can be achieved simultaneously with immobilization of the biocompatible polymer. For sterilization, it is preferable that the substance permeability of radiation is high to some extent, and therefore, among the above radiations, β rays, γ rays, X-rays, and electron beams are more preferable.

In addition, an antioxidant may be used to suppress the crosslinking reaction of the biocompatible polymer by irradiation with radiation. The antioxidant means a substance having a property of easily giving electrons to other molecules. Examples of antioxidant include, but are not limited to, water-soluble vitamins such as vitamin C, polyphenols, and alcohol-based solvents such as methanol, ethanol and propanol. These antioxidants may be used singly, or in combination of two or more thereof. When the safety needs to be considered, an antioxidant having low toxicity such as ethanol or propanol is suitably used.

### Hollow Fiber Membrane Module

The hollow fiber membrane module preferably contains the hollow fiber membrane.

Examples of the method of modularizing the hollow fiber membrane include a method in which the hollow fiber membrane is fixed to a case while being centrifuged, and a method in which the hollow fiber membrane is formed into a U-shape and only the opening side of the hollow fiber membrane is fixed to the case. Although not particularly limited, an example is as follows. First, a hollow fiber membrane is cut into a required length, a required number of the hollow fiber membranes are bundled and then put into a cylindrical case. Then, the cylindrical case is temporarily capped at both the ends, and a potting material is put into both the end portions of the hollow fiber membrane. At this time, a method of putting the potting material during rotating the module with a centrifuge is preferable because the potting material can be uniformly filled. After the potting material is solidified, the hollow fiber membrane is cut at both end portions so as to be opened at both ends. A liquid to be treated inlet (hereinafter, “header”) is attached, and the headers and the nozzle part of the case are plugged to obtain a hollow fiber membrane module.

The hollow fiber membrane is preferably subjected to a crimping treatment because retention of a liquid to be treated (such as blood) in the hollow fiber membrane module can be prevented. That is, the hollow fiber membrane module preferably contains the hollow fiber membrane subjected to crimping treatment.

When the hollow fiber membrane module is excessively large, handling becomes complicated, and therefore an effective membrane area is preferably 0.1 m² or more and 3.0 m² or less. The effective membrane area is more preferably 0.15 m² or more, still more preferably 0.2 m² or more, and particularly preferably 0.25 m² or more from the viewpoint of treatment capacity. On the other hand, the effective membrane area is more preferably 2.0 m² or less, still more preferably 1.5 m² or less, and particularly preferably 1.0 m² or less because the module size can be reduced and handling is easy.

The dimensions of the hollow fiber membrane module are not particularly limited, but the inner diameter is preferably 1.0 cm or more and 9.0 cm or less, more preferably 1.5 cm or more and 7.0 cm or less, and still more preferably 2.0 cm or more and 5.5 cm or less from the viewpoint of the preferred effective membrane area range and the ease of handling of the module.

The effective length of the hollow fiber membrane inside the hollow fiber membrane module is also preferably 10.0 cm or more and 40.0 cm or less, more preferably 15.0 cm or more and 35.0 cm or less, and still more preferably 20.0 cm or more and 30.0 cm or less from the viewpoint of an effective membrane area range and handleability of the module.

The ratio of the volume of the hollow fiber membrane to the volume inside the module, that is, a filling rate is preferably 30% or more and 70% or less, more preferably 35% or more and 65% or less, still more preferably 40% or more and 60% or less, and particularly preferably 45% or more and 55% or less, from the viewpoint of ease of flow of the liquid to be treated in the module and effective use of the module volume.

The filling rate (%) can be determined by the following equation.

$$( Filling rate ) = ( sectional area of hollow fiber membrane ) \times ( number of hollow fiber membranes ) / ( module cross - sectional area ) \times 100 \\ where : \\ ( sectional area of hollow fiber membrane ) = {\{ ( outer diameter of hollow fiber membrane ) / 2 \}}^{2} \times circular constant π \\ ( module cross - sectional area ) = {\{ ( module inner diameter ) / 2 \}}^{2} \times circular constant π$$

In the hollow fiber membrane, since the average pore size of the inner surface is 0.50 μm or more and 3.00 μm or less, when the inner surface is used as a blood-contacting surface, the platelet adhesion suppressing effect can be greatly exhibited. That is, the structure of the hollow fiber membrane module is preferably a hollow fiber membrane module including a liquid to be treated inlet and a filtrate outlet, the liquid to be treated passing from the inside to the outside of the hollow fiber membrane according to the present invention and flowing out from the filtrate outlet.

For the same reason, also in a second aspect of the hollow fiber membrane, the average pore size of the inner surface is preferably 0.50 μm or more and 3.00 μm or less.

The hollow fiber membrane can be suitably used for blood purification. That is, a hollow fiber membrane used for blood purification is preferable, and a hollow fiber membrane module containing the hollow fiber membrane is preferable. This is because, as described above, the hollow fiber membrane exhibits high blood compatibility due to its structure.

As described above, the hollow fiber membrane module may be a U-shaped module. That is, one of preferred embodiments is a U-shaped hollow fiber membrane module containing the hollow fiber membrane. The U-shaped module is a module having a structure in which the hollow fiber membrane is folded back in a U shape, both ends of the hollow fiber membrane bundle are bonded to a case with a resin, and both ends of the hollow fiber membrane are opened. The U-shaped module is easy to handle, and is suitably used for research, test applications, and inspection applications. In addition, a method of producing a vesicle-containing solution, in which a vesicle-containing solution is obtained from a liquid to be treated by using the U-shaped hollow fiber membrane module, is preferred.

### Vesicle and Vesicle-containing Solution

The type of the vesicle is not particularly limited, and the vesicle is preferably a vesicle having, as a surface marker, at least one selected from the group consisting of phosphatidylserine, CD9, CD63, and CD81.

Among the markers described above, phosphatidylserine and CD9 are surface markers of vesicles that are suitably used. Phosphatidylserine is a type of phospholipid, and is present only inside a cell membrane, but is also present outside a vesicle. CD9 is a type of four-pass transmembrane protein called tetraspanin.

The vesicle in the measurement sample liquid can be detected and quantified by an ELISA (Enzyme-Linked Immuno-Sorbent Assay) method using phosphatidylserine and CD9 as markers. In this instance, it is preferable to use an antibody that binds to phosphatidylserine as the plate-side capture antibody and an antibody that binds to CD9 as a labeled detection antibody.

The vesicle-containing solution is purified by the hollow fiber membrane module.

The solvent used in the vesicle-containing solution is not particularly limited, and examples thereof include water, ethanol, and dimethyl sulfoxide. These solvents may be used in combination. In addition, since the vesicle is a biological sample, it is more preferable to use a buffer solution containing water as a main component as a solvent. Examples of the buffer solution include a phosphate buffer solution, a phosphate buffered saline solution, an acetate buffer solution, a Tris buffer solution, and a citrate buffer solution.

In addition, from the viewpoint of preventing deterioration, the vesicle-containing solution preferably contains a vesicle stabilizer having a weight average molecular weight of 10,000 or more and 1,000,000 million or less. The vesicle stabilizer in this instance is preferably a polymer of at least one unit selected from the group consisting of 2-methacryloyloxyethyl phosphorylcholine, vinyl alcohol, vinyl pyrrolidone, methoxyalkylene glycol monomethacrylate, and 2-hydroxyethyl methacrylate. Among them, a polymer of 2-methacryloyloxyethyl phosphorylcholine is particularly preferable.

The size of the vesicle contained in the vesicle-containing solution is not particularly limited, and a vesicle having a diameter of 300 nm or less is less likely to precipitate, and the quality of the vesicle-containing solution containing the vesicle is likely to be stabilized, which is preferable. That is, the vesicle-containing solution contains vesicles having a size distribution, and is preferably a vesicle-containing solution containing 99% or more of the vesicles having a diameter of 30 nm or more and 300 nm or less on a number basis. Furthermore, the vesicle content in a diameter of 30 nm or more and 300 nm or less is more preferably 99.9% or more.

The vesicle-containing solution can be suitably used for, for example, examination, treatment, beauty, and anti-aging. Since the vesicle contains an information transfer substance such as a protein or a nucleic acid, a lot of information about the state in the body can be obtained, and the vesicle can be an effective test target. For example, it is known that cancer cells release a vesicle that acts to weaken an attack on an immune cell that intends to attack itself, and the presence or absence of cancer can be examined by a vesicle-containing solution obtained by purifying a body fluid collected from a subject. In addition, since the activity of the cell can be changed by the information transfer substance in the vesicle, it can also be used for treatment. For example, contrary to the above example, a method is considered in which a vesicle instructing an immune cell to attack a cancer cell acts on the immune cell. Furthermore, by promoting decomposition of waste products and metabolism in cells, effects of beauty and anti-aging can also be expected. Suitable methods for administering the vesicle-containing solution include injections and drips for therapeutic and anti-aging purposes, and application as a cosmetic for cosmetic purposes.

### EXAMPLES

Our membrane, module and solution will be illustrated below with reference to examples, but it should be understood that this disclosure is not construed as being limited thereto.

### (1) Preparation of Hollow Fiber Membrane Module

10 hollow fiber membranes were filled in a case having a diameter of about 5 mm and a length of about 17 cm. Both ends were potted with an epoxy resin-based chemically reactive adhesive “Quick Mender” (registered trademark) manufactured by Konishi Co., Ltd., and then the end surface was cut and opened to prepare a hollow fiber membrane module having an effective length of 12 cm.

### (2) Measurement of Particle Permeability

The particle permeability of the hollow fiber membrane was measured using commercially available polystyrene latex beads (hereinafter, “LB”) having average particle sizes of 0.15 μm, 0.20 μm, and 1.00 μm (“Sulfate Latex Beads” manufactured by Thermo Fisher). The relationship between the concentration and turbidity of the LB suspension was previously calculated from the absorbance at a wavelength of 260 nm measured by ultraviolet-visible spectrometry. The turbidity of the filtrate filtered through the hollow fiber membrane was measured, and the particle permeability was determined from the following equation. The liquid to be treated at this time was diluted with pure water to have an LB concentration of 0.02 mass % (200 ppm). In the measurement, two hollow fiber modules were prepared, treated water was caused to flow from the inside to the outside of the hollow fiber membrane at a pressure of 13.3 kPa, an average value of n=2 was calculated, and a value obtained by rounding off the average value to the one decimal place was used as measurement data.

$$particle permeability ( \% ) = \{ ( turbidity of filtrate ) / ( turbidity before treatment ) \} \times 100$$

### (3) Measurement of Water Permeability

The hollow fiber membrane of the hollow fiber membrane module prepared by the above method (1) and the inside of the module were washed with distilled water for 30 minutes. The filtrate volume per unit time was measured by applying a water pressure of 150 mmHg to the interior of the hollow fiber membrane and measuring the amount of water flowing out of the hollow fiber membrane. The water permeability (UFR) was calculated by the following equation, and a value obtained by rounding off the value to the one decimal place was used as measurement data.

$$UFR ( mL / h / mm Hg / {m}^{2} ) = Qw / ( P \times T \times A )$$

Qw is filtration amount (mL), T is outflow time (h), P is pressure (mmHg), and A is inner surface area (m²) of the hollow fiber membrane.

The inner surface area A of the hollow fiber membrane was determined by multiplying the inner diameter of the hollow fiber by the circular constant π and the length of the part effective for the measurement among the hollow fibers used for the water permeability measurement.

### (4) Measurement of Hydrophilic Polymer Content

The nitrogen atom content of the hollow fiber membrane was measured by elemental analysis to calculate the hydrophilic polymer content in the hollow fiber membrane. The hollow fiber membrane was freeze-pulverized, and then dried under reduced pressure at normal temperature (25° C.) for 2 hours to obtain a measurement sample. A measuring apparatus and measuring conditions were as follows. The measurement was performed three times, and the average value thereof was used as the measured value.
Measuring apparatus: Nitrogen micro-detector ND-100 (manufactured by Mitsubishi Chemical) Electric furnace temperature (horizontal reactor)
Thermal decomposition section: 800° C.
Catalyst section: 900° C.
Main O₂ flow rate: 300 mL/minute
Sub O₂ flow rate: 300 mL/minute
Ar flow rate: 400 mL/minute
Sens.: Low

The molar mass of the nitrogen atom is 14 g/mol, and the molar mass of the repeating unit of PVP is 111 g/mol. Therefore, the hydrophilic polymer content (mass %) was calculated by the following equation using the nitrogen atom amount (μg/g) contained in the hollow fiber membrane per unit mass obtained by elemental analysis, and a value rounded off to the two decimal place was used.

$$Hydrophilic polymer content ( mass \% ) =$$

$$Nitrogen atom amount contained in hollow fiber membrane \times$$

$$111 / 14 \times 100$$

In the example when the main component of the hollow fiber membrane is polysulfone and the hydrophilic polymer is PVP or PVP and a vinyl propionate/vinyl pyrrolidone copolymer has been described, but the hydrophilic polymer content can be determined in the same manner by appropriately changing the element to be quantified by elemental analysis and the calculation formula depending on the type of the polymer to be used.

### (5) Measurement of Proportion of Insoluble Component

The proportion of insoluble components was measured as follows. Approximately 1 g of the dried hollow fiber membrane was weighed into an Erlenmeyer flask, 40 mL of DMAc was added thereto as a good solvent for the hollow fiber membrane, and the mixture was stirred with a stirrer at room temperature (25° C.) for 2 hours. Next, centrifugation was performed at 2,500 rpm to precipitate insoluble components, and the supernatant was removed. A series of operations of adding 10 mL of DMAc to the obtained insoluble component, washing by stirring again with a stirrer, and removing the supernatant after centrifugation was repeated 3 times. Finally, the supernatant was removed, and the obtained insoluble component was freeze-dried. The mass of the insoluble component was measured, and the ratio of the insoluble component to the total mass of the hollow fiber membrane was calculated by the following equation. The proportion of insoluble components was rounded off to the two decimal place.

$$Ratio ( mass \% ) of insoluble component = mass of insoluble component / mass of hollow fiber membrane \times 100$$

In the example when the main component of the hollow fiber membrane is polysulfone and the hydrophilic polymer is PVP or PVP and a vinyl propionate/vinyl pyrrolidone copolymer has been described, but the proportion of insoluble components can be determined in the same manner by appropriately changing the good solvent for dissolving the hollow fiber membrane depending on the type of the polymer to be used.

### (6) TOF-SIMS measurement

In a hollow fiber membrane, the membrane was trimmed and cut to a semi-cylindrical shape with a single-edged blade, and measurement was performed at three different sites on the inner or outer surface of the hollow fiber membrane. The measurement sample was rinsed with ultrapure water, then dried at room temperature and 0.5 Torr for 10 hours, and then subjected to measurement. A measuring apparatus and conditions were as follows.
Measuring apparatus: TOF. SIMS 5 (manufactured by ION-TOF Company)
Primary ion: Bi³⁺⁺
Primary ion acceleration voltage: 30 kV
Pulse width: 5.9 ns
Secondary ion polarity: negative
Number of scans: 64 scan/cycle
Cycle Time: 140 μs
Measurement range: 200 μm×200 μm
Mass range (m/z): 0 to 1500

The presence or absence of carboxylic acid ions on the surface of the hollow fiber membrane was confirmed from the obtained mass m/z spectrum. When the carboxylic acid ion strength is 0.4% or less based on the total secondary ion strength, the value of the carboxylic acid ion strength is judged ascribable to noise, and it is determined that there is no carboxylic acid.

### (7) Measurement of Inner Diameter and Membrane Thickness of Hollow Fiber Membrane

The inner diameter and membrane thickness of the hollow fiber membrane were measured by the following method. That is, the average a of membrane thickness was obtained by measuring each of the membrane thickness of 16 randomly selected hollow fiber membranes with a lens of 1,000 magnifications of Microwatcher (VH-Z100 manufactured by KEYENCE Corporation). The inner diameter of the hollow fiber membrane was calculated by the following equation. Regarding for the outer diameter of the hollow fiber membrane, the average, obtained by measuring the outer diameter of each of 16 randomly selected hollow fiber membranes with a laser displacement gage (for example, LS5040T manufactured by KEYENCE Corporation), was used.

$$hollow fiber membrane inner diameter ( µm ) = average of hollow fiber membrane outer diameter - ( 2 \times average a of membrane thickness )$$

### (8) Measurement of Average Pore Size and Average Depth of Pores on Surface of Hollow Fiber Membrane

A hollow fiber membrane that was wetted with water for 5 minutes, then frozen with liquid nitrogen, and freeze-dried was used as a measurement sample. The hollow fiber membrane was cut in half in axial direction to expose the interior surface. Using a scanning electron microscope (SEM) (S-5500 manufactured by Hitachi High-Technologies Corporation), the inner surface of the hollow fiber membrane was observed at a magnitude of 1,500, and the outer surface was observed at a magnitude of 3,000, and the images were captured in a computer. The size of the captured image was 640 pixels×480 pixels. The number of pores and the area of each pore were measured using image processing software (ImageJ (version 1.52), developer National Institutes of Health) for pores in the range of 40 μm×40 μm on the inner surface of the hollow fiber membrane and for pores in the range of 20 μm×20 μm on the outer surface. The measurement in the range of 40 μm×40 μm or 20 μm×20 μm was repeated until the total number of measured pores reached at least 50, and data was added. When the pores were observed doubly in the depth direction, the exposed portion of the deeper pore was measured. When a part of the pore was out of the measurement range (visual field of SEM image), the pore was excluded. The SEM image was binarized to obtain an image wherein a hollow part is black and the structure part is white. If it was not possible to clearly binarize the hollow parts and structure parts due to contrast differences in the analysis image, the hollow parts were painted black before image processing, the pores were fitted into a circular shape, and the pore size was measured. At this time, to cut noise, pores having an area in which the number of continuous pixels is 5 pixels or less were excluded from the data.

The average pore size was calculated from the measurement results using the following equation and rounded off to the three decimal place.

$$Average pore size ( µm ) = {\{ ( S / a ) / π \}}^{1 / 2} \times 2$$

a is the number of pores measured, S is the sum of the areas of the pores measured (μm²), and π is a circular constant.

The average depth of pores in the inner surface of the hollow fiber membrane was measured as follows. The hollow fiber membrane was cut into the above-mentioned semi-cylindrical shape, and the inner surface of the hollow fiber membrane with the inner surface exposed was observed at a magnification of 150 using a laser microscope (VK-9710 manufactured by KEYENCE CORPORATION). The obtained results were analyzed by shape analysis software “VK ANALYZER” equipped with a laser microscope to calculate the average depth of pores in the inner surface.

### (9) Measurement of Open Pore Ratio

In the same manner as in the above (8), using a scanning electron microscope (SEM) (S-5500 manufactured by Hitachi High-Tech Corporation), the inner surface of the hollow fiber membrane was observed at a magnitude of 1,500 and the outer surface was observed at 3,000 to take the image into the computer. The size of the captured image was 640 pixels×480 pixels. The SEM image was cut into a range of 40 μm×40 μm for the inner surface and a range of 20 μm×20 μm for the outer surface, and image analysis was performed using image processing software. A threshold was determined so that the structure part had high brightness and the other parts had low brightness by a binarizing process, and an image in which the high brightness part was white and the low brightness part was black was obtained. When the structure part and the other parts were not able to be divided due to the difference in contrast in the image, the image was cut at the part having similar contrast ranges, the cut images were each binarized, and then connected back to one image. Alternatively, the part other than the structure part can be filled in black to perform image analysis. The image contained noise, and the noise was not able to be distinguished from pores in the low brightness part having continuous pixels of 5 or less. Thus, the low brightness part was taken as the high brightness part as a structure part. As a method of eliminating noise, the low brightness part having continuous pixels of 5 or less is removed on the measurement of the number of pixels. Alternatively, the noise part can be filled in white. The number of pixels in the low brightness part was measured, and the percentage of the number of pixels in the low brightness part to the total number of pixels for forming an analysis image was calculated and taken as the open pore ratio. The same measurement was performed on five images, an average value was calculated, and a value obtained by rounding off the average value to the two decimal place was used.

$$Open pore ratio ( \% ) = S / A \times 100$$

A: Area of measurement range, S: Sum of areas of measured pores

### (10) Measurement of Thickness of Dense Layer

A hollow fiber membrane that was wetted with water for 5 minutes, then frozen with liquid nitrogen, quickly folded, and freeze-dried was used as an observation sample. A cross section of the hollow fiber membrane was observed at a magnification of 2,000 using a SEM (S-5500 manufactured by Hitachi High-Tech Corporation) to take the image into the computer. The size of the captured image was 640 pixels×480 pixels. When the pores in the cross section were observed with SEM and were blocked, the sample preparation was carried out again. The closing of the pore may be caused by deformation of the hollow fiber membrane in the stress direction during the cutting.

The obtained SEM image was subjected to image analysis using image processing software. The analysis range may be any length as long as the entire membrane thickness fits. When the entire membrane thickness was not settled in the observation field of the measurement magnification, two or more SEM images were synthesized. A threshold was determined so that the structure part had high brightness and the other parts had low brightness by a binarizing process, and an image in which the high brightness part was white and the low brightness part was black was obtained. When the structure part and the other parts were not able to be divided due to the difference in contrast in the image, the image was cut at the part having similar contrast ranges, the cut images were each binarized, and then connected back to one image. Alternatively, the part other than the structure part can be filled in black to perform image analysis. When the pores were observed doubly in the depth direction, the shallower pore was measured. When a part of the pore was out of the measurement range, the pore was excluded. The image contained noise, and the noise was not able to be distinguished from pores in the low brightness part having continuous pixels of 5 or less. Thus, the low brightness part was taken as the high brightness part as a structure part. As a method of eliminating noise, the low brightness part having continuous pixels of 5 or less is removed on the measurement of the number of pixels. Alternatively, the noise part can be filled in white. The number of pixels of a scale bar indicating a known length in the image was measured, and the length per pixel was calculated. The number of pixels of the detected pore was measured, and the pore area was obtained by multiplying the number of pixels of the pore by the square of the length per pixel. The diameter of a circle corresponding to the pore area was calculated by the following equation and taken as the pore size. The pore area having a pore size of 0.5 μm is 0.2 μm².

$$Pore size ( µm ) = 2 \times {( pore area / π )}^{1 / 2}$$

In the binarized image, pores having a pore size of 0.5 μm or more, that is, pores having a pore area of 0.2 μm² or more were specified, a perpendicular line was drawn from the outer surface toward the inner surface of the hollow fiber membrane, and among the layers in which pores having a pore area of 0.2 μm² or more were not observed, the thickest region was defined as a dense layer, and the thickness of the dense layer was measured. 10 points were measured in the same image. Further, the same measurement was performed on 3 images, the average value of 30 pieces of measurement data in total was calculated, and the value obtained by rounding off the average value to the two decimal place was taken as the dense layer thickness.

### (11) Measurement of Vesicle Permeability

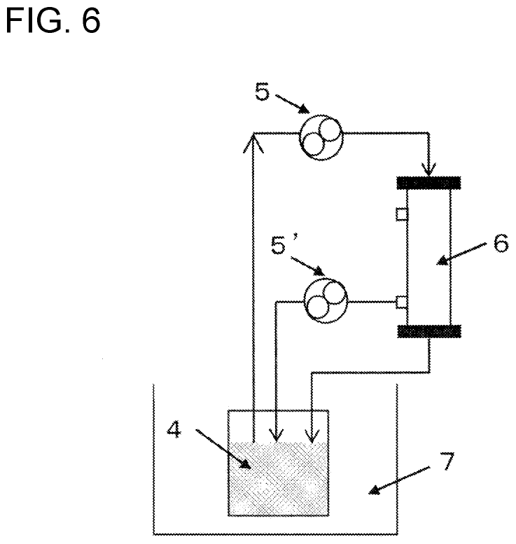

For the test, the hollow fiber membrane module prepared by the above method (1) was used. An outline of the vesicle permeability measurement system is shown in FIG. 6. 20 mL of commercially available human serum (Cosmo Bio Co., Ltd., product code: 12181450) was placed in a container to obtain a human serum pool 4, and the human serum pool 4 was kept at 37° C. using a thermostatic bath 7. The liquid passed through the inside of the hollow fiber membrane in a hollow fiber membrane module 6 at a rate of 0.5 mL/min using a circulation pump 5, and the hollow fiber membrane module 6 was brought into a circulation state. At the same time, filtration was performed from the outside of the hollow fiber membrane at a rate of 0.15 mL/min using a filtration pump 5′. At this time, a filtrate was returned to the human serum pool 4. The circulation time was set to 120 minutes, and the human serum and the filtrate in the container were appropriately sampled, and the vesicle concentration was measured by the sandwich ELISA method described below. The vesicle permeability was calculated by the following equation, and the value obtained by rounding off the one decimal place was taken as the vesicle permeability.

$$Vesicle permeability ( \% ) = ( vesicle concentration in filtrate /$$

$$vesicle concentration of human serum in container ) \times 100$$

For the measurement of the vesicle concentration of human serum in the filtrate and in the container, when a vesicle having CD9 and CD63 as surface markers was measured, “CD9/CD63 Exosome ELISA kit (trade name)” (manufactured by Cosmo Bio Co., Ltd.) was used. First, the sample solution was placed in a plate on which the anti-CD9 antibody of the kit was immobilized, and allowed to stand for 2 hours to immobilize the CD9-positive vesicle on the plate. After washing each well, an HRP-labeled anti-CD63 antibody (detection antibody) was added to each well and allowed to stand for 2 hours to bind the detection antibody to a CD63-positive vesicle. After washing each well again, a substrate solution was added to each well and reacted for 20 minutes to develop color. A reaction stop solution was added to stop the coloring reaction, and then the absorbance of each well at a wavelength of 450 nm was measured with a plate reader to determine the vesicle concentration. At this time, a CD9/CD63 fusion protein attached to the kit was used as a standard substance for preparing a calibration curve.

Vesicles with surface markers PS and CD9 were quantified by a similar sandwich ELISA. Using a plate on which an anti-phosphatidylserine antibody was immobilized (“‘PS Capture’ (registered trademark) Exosome ELISA Kit (Streptavidin HRP) (trade name)” 96 well plate, manufactured by FUJIFILM Wako Pure Chemical Corporation), a shaking reaction was performed at 500 rpm for 2 hours. After washing, a shaking reaction was carried out for 1 hour using a biotin-conjugated anti-CD9 antibody (manufactured by FUJIFILM Wako Pure Chemical Corporation) as a detection antibody. After washing, HRP-labeled streptavidin was shaken for 2 hours. After washing, the substrate solution was added, and the mixture was allowed to stand for 20 minutes. Then, absorbances at a dominant wavelength of 450 nm and a secondary wavelength of 620 nm were measured, and the concentration was calculated using a value obtained by subtracting the absorbance at 620 nm from the absorbance at 450 nm. At this time, a vesicle-containing solution purified from volunteer human fresh plasma using magnetic beads (“‘MagCapture’ (registered trademark) Exosome Isolation Kit PS (trade name)”, manufactured by FUJIFILM Wako Pure Chemical Corporation) was used for creating a calibration curve.

Pressure measurement was also performed on the hollow fiber membrane module inlet side and the filtration side during circulation. Based on the pressure immediately after the start of circulation, the amount of change in the filtration side pressure at 30 minutes and 120 minutes from the start of circulation was calculated.

### (12) Test of Attachment of Platelets

A double-faced tape was attached to a circular plate made of polystyrene having a diameter of 18 mm, and a hollow fiber membrane was fixed thereto. The attached hollow fiber membrane was shaved and cut into a semi-cylindrical shape with a single edged knife to expose the inner surface of the hollow fiber membrane. When the inner surface of the hollow fiber membrane has dirt, scratches, and folds, platelets may attach there, which may result in incorrect evaluation. Thus, the hollow fiber membrane with dirt, scratches and folds was excluded from the measurement target. The circular plate was attached to a polypropylene tube cut into a cylindrical shape and having a diameter of 18 mm so that the surface to which the hollow fiber membrane was attached was inside the cylinder, and the gap was filled with parafilm. The inside of the cylindrical tube was washed with physiological saline, and then filled with physiological saline. A test solution was prepared by adding heparin sodium at 50 U/mL to venous blood (number of red blood cells: 4,500,000 to 5,000,000/mm³, number of white blood cells: 5,000 to 8,000/mm³, number of platelets: 200,000 to 500,000/mm³) collected from a healthy human. After discarding the physiological saline in the cylindrical tube, 1.0 mL of the test liquid was added to the cylindrical tube within 30 minutes after blood collection, and the tube was shaken at a rotation speed of 700 rpm at 37° C. for 1 hour. Thereafter, the hollow fiber membrane was washed with 10 mL of physiological saline, 2.5 vol % glutaraldehyde/physiological saline was added, and the adhered platelets were immobilized on the hollow fiber membrane. After a lapse of 1 hour or more, it was washed with 20 mL of distilled water. The washed hollow fiber membrane was dried under reduced pressure at a room temperature (25° C.) and 0.5 Torr for 10 hours. The hollow fiber membrane was attached to a stage of a scanning electron microscope with double-faced tape. Thereafter, a thin film of Pt-Pd was formed on the surface of the hollow fiber membrane by sputtering to obtain a platelet-attached sample. The SEM image of the inner surface of the obtained platelet-attached sample was observed with a scanning electron microscope (S-3000 Type H, manufactured by Hitachi Science Systems, Ltd.) at a magnification of 1,500 times, and the number of platelets in one visual field (5.2×103 μm²) was counted. The number of observation fields was set to 20 fields per sample, and the average value thereof was obtained. When the number of particles exceeded 50/5.2×103 μm² in one visual field, it was determined that measurement was impossible. In addition, a part of the end of the hollow fiber in the longitudinal direction was excluded from the measurement target of the number of attached platelets because it was easy to form a blood pool. The test of this procedure was performed using test liquids derived from three different humans, and the results were averaged and rounded off to the two decimal place to obtain the number of attached platelets.

### Example 1

13 mass % of polysulfone (“Udel” (registered trademark) P-3500 manufactured by SOLVAY) and 7.4 mass % of PVP (Povidone (PLASDONE) K90 manufactured by ASHLAND LCC) were added to a solvent made of 76.6 mass % of DMAc and 3 mass % of water, and the mixture was heated and dissolved at 90° C. for 14 hours to obtain a membrane formation dope solution. This membrane formation dope solution was ejected through an orifice-type double cylindrical die having an outer diameter of 1.0 mm/an inner diameter of 0.7 mm adjusted to 37° C. At the same time, as a core solution, a solution containing 92 mass % of DMAc and 8 mass % of water was ejected from the inner tube. After passing through a dry section having a length of 70 mm set at a dew point of 28° C., the solutions were immersed in a coagulation bath at 80° C. containing water to be coagulated. Further, the coagulated resultant was washed with warm water in a water washing bath at 80° C. and then wound around a skein frame at a speed of 30 m/min to obtain a hollow fiber membrane in a wet state. The inner diameter of the obtained hollow fiber membrane was 317 μm, and the membrane thickness was 50 μm.

The obtained hollow fiber membrane in a wet state was subdivided by cutting the hollow fiber membrane to a length of 0.4 m, and immersed in a warm water bath at 90° C. for 3 hours to be washed with warm water, and was then dried at 100° C. for 10 hours, and further heated at 170° C. for 5 hours by a dry heat dryer to perform a crosslinking treatment, thereby obtaining a hollow fiber membrane 1. Further, a hollow fiber membrane module 1 was prepared by the above method (1). Data obtained in the measurement of the above (2) to (11) are shown in Table 1.

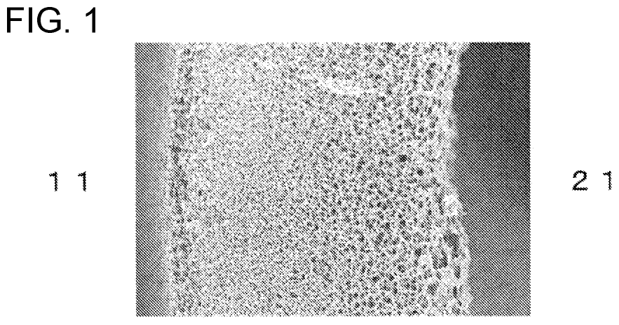

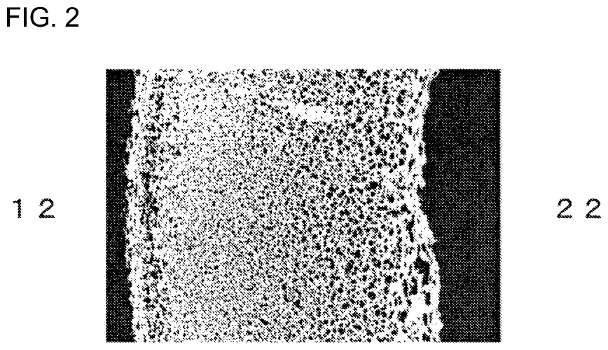

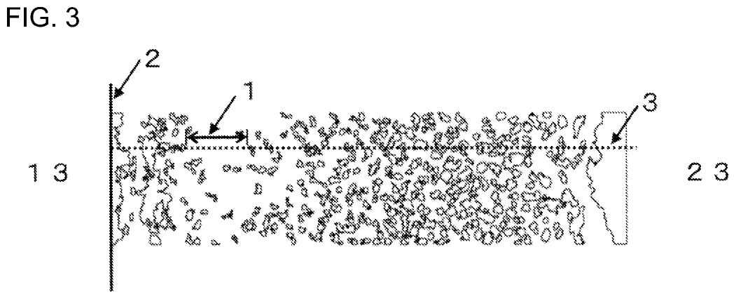

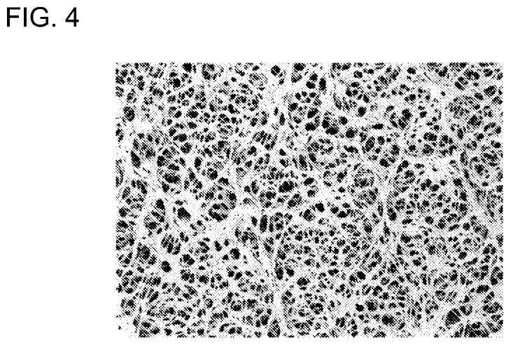

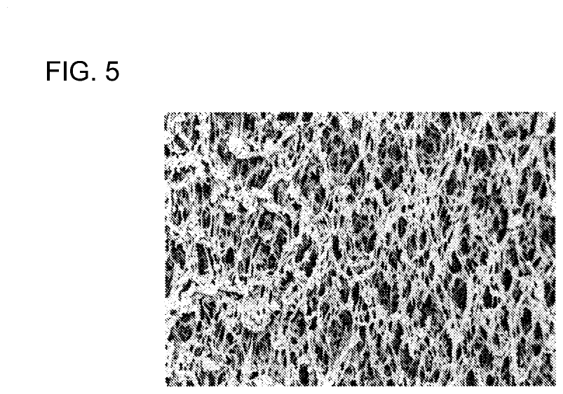

Regarding the results of observing the cross section of the hollow fiber membrane with SEM, a sectional SEM image is shown in FIG. 1, an inner surface SEM image is shown in FIG. 4, and an outer surface SEM image is shown in FIG. 5. It was an asymmetric three-dimensional network structure having a dense layer in the vicinity of the outer surface. That is, the outer surface is a surface 1, and the inner surface is a surface 2. The thickness of the dense layer was 4.5 μm. FIG. 2 illustrates a binarized image at the time of analysis. FIG. 3 illustrates a view obtained by deleting a pore having a pore size of 0.5 μm or less. In FIG. 3, a left vertical line is a tangent line 2 drawn on the outer surface of the hollow fiber membrane. A horizontal line of a dotted line in FIG. 3 is a perpendicular line 3 to a tangent line drawn on the outer surface. The thickness of the dense layer was determined by measuring a distance at which no pore was recognized on the perpendicular line 3. Reference numeral 1 in FIG. 3 is an example 1 of the recognized distance between the pores, that is, the thickness of the dense layer. The obtained hollow fiber membrane module 1 had high particle permeability at particle sizes of 0.15 μm and 0.20 μm, small changes in pressure due to clogging, and high vesicle permeability. In FIGS. 1 to 3, the left side of the image is the outer surface sides 11, 12, and 13 of the hollow fiber membranes, and the right side is the inner surface sides 21, 22, and 23.

### Example 2

A hollow fiber membrane 2 was obtained in the same experiment as in Example 1 except that the membrane formation dope solution was ejected at a temperature of 40° C. and the temperature of the coagulation bath was 90° C. The inner diameter of the obtained hollow fiber membrane was 313 μm, and the membrane thickness was 71 μm. The hollow fiber membrane module 2 was obtained by the same operation as in Example 1. Data obtained in the measurement of the above (2) to (11) are shown in Table 1.

As a result of observing the cross section of the hollow fiber membrane with SEM, it was an asymmetric three-dimensional network structure having a dense layer in the vicinity of the outer surface. That is, the outer surface is a surface 1, and the inner surface is a surface 2. The obtained hollow fiber membrane module 2 had high particle permeability at particle sizes of 0.15 μm and 0.20 μm as in Example 1. The change in pressure due to clogging was also small, and high vesicle permeability was exhibited.

### Comparative Example 1

A hollow fiber membrane 3 was obtained in the same experiment as in Example 1 except that 15 mass % of polysulfone, 7 mass % of PVP, 75.4 mass % of DMAc, and 2.6 mass % of water were used in the membrane formation dope solution. The inner diameter of the hollow fiber membrane was 300 μm, and the membrane thickness was 80 μm. The hollow fiber membrane module 3 was obtained by the same operation as in Example 1. Data obtained in the measurement of the above (2) to (11) are shown in Table 1.

As a result of observing the cross section of the hollow fiber membrane with SEM, it was an asymmetric three-dimensional network structure having a dense layer in the vicinity of the outer surface. That is, the outer surface is a surface 1, and the inner surface is a surface 2. In the obtained hollow fiber membrane module 3, the particle permeability of the particle size of 0.15 μm was low, the permeability of the vesicle was low, the filtration pressure was large, the filtration pressure became negative pressure, and occurrence of clogging was observed.

### Comparative Example 2

A hollow fiber membrane module 4 was obtained in the same manner as in Example 1 using a plasma separator “Plasma flow” (registered trademark) manufactured by Asahi Kasei Medical Co., Ltd. having a nominal pore size of 0.3 μm. Data obtained in the measurement of the above (2) to (11) are shown in Table 1.

As a result of observing the cross section of the hollow fiber membrane with SEM, the hollow fiber membrane had a uniform structure rather than an asymmetric three-dimensional network structure. The obtained hollow fiber membrane module 4 had a low particle permeability with a particle size of 0.15 μm. The permeability was low, the filtration pressure was large and the filtration pressure was negative, and occurrence of clogging was observed.

### Example 3

An aqueous solution obtained by dissolving a vinylpyrrolidone/vinyl propanoate random copolymer (mole fraction of vinyl propanoate unit: 40%, number average molecular weight: 16,500) was dissolved at a concentration of 100 ppm and an ethanol concentration of 1,000 ppm in the hollow fiber membrane module 1 obtained in Example 1 was passed from the inside to the outside of the hollow fiber membrane to coat the entire membrane. Subsequently, a y-ray of 25 kGy was irradiated to obtain a hollow fiber membrane module 5. Data obtained in the measurement of the above (2) to (11) are shown in Table 1.

As a result of observing the cross section of the hollow fiber membrane with SEM, it was an asymmetric three-dimensional network structure having a dense layer in the vicinity of the outer surface. That is, the outer surface is a surface 1, and the inner surface is a surface 2. The obtained hollow fiber membrane module 5 had high particle permeability at particle sizes of 0.15 μm and 0.20 μm as in Example 1. In addition, in the measurement of vesicle permeability, clogging due to adhesion of proteins and the like was suppressed by the protein adhesion suppressing effect of the biocompatible polymer, the change in pressure was the smallest (in Examples 1 and 2, the negative pressure was 0.5 kPa or more, whereas in Example 3, there was substantially no change), and high vesicle permeability was exhibited.

Further, the hollow fiber membrane module 5 was disassembled to obtain a hollow fiber membrane coated with a vinylpyrrolidone/vinyl propanoate random copolymer. The average depth of pores in the inner surface of the obtained hollow fiber membrane was measured and found to be 1.62 μm. In addition, when the test of platelet attachment was performed on the inner surface, the number of attached platelets was 7/5.2×103 μm². It is considered that the reduction of the average depth of the pores in the inner surface of the hollow fiber membrane is one of the causes of the development of such excellent blood compatibility.

The difference (73%) in permeability of a vesicle having phosphatidylserine and CD9 as surface markers at a circulation time of 120 minutes between Example 2 and Comparative Example 2 is larger than the difference (40%) in permeability of a vesicle having CD9 and CD63 as surface markers, and it can be seen that the hollow fiber membrane is particularly excellent in separation of a vesicle having phosphatidylserine and CD9 as surface markers. The reason why such a difference occurs depending on the surface marker to be used is not clear, but can be presumed. That is, as described above, the hollow fiber membrane has high particle permeability of 0.20 μm, and is excellent not only in permeability of exosomes but also in permeability of microvesicles.

### Comparative Example 3

“Toraylight” (registered trademark) NV manufactured by TORAY MEDICAL CO., LTD. was disassembled, and the average pore size of the inner surface of the obtained hollow fiber membrane was 0.005 μm. The average depth of the pores in the inner surface was measured and found to be 0.01 μm. When the test of platelet attachment was performed on the inner surface, the number of attached platelets was 2/5.2×103 μm². A hollow fiber membrane module 6 was obtained by the above method (1). The hollow fiber membrane module 6 was subjected to 0.04 μm particle permeability measurement, but no permeation was observed. Such particle permeability does not allow permeation of particles having a particle size of 0.15 μm. That is, the average depth of pores in the inner surface of the hollow fiber membrane was shallow, and the hollow fiber membrane was excellent in blood compatibility, but was insufficient in particle permeability.

### Comparative Example 4

“Mascure” (registered trademark) ascitic fluid filtration filter manufactured by SB-KAWASUMI LABORATORIES, INC. was disassembled, and the average pore size of the inner surface of the obtained hollow fiber membrane was 3.39 μm. The average depth of the pores in the inner surface was measured and found to be 7.08 μm. When the test of platelet attachment was performed on the inner surface, a large number of platelets were attached and thus measurement was impossible. It can be seen that when the average pore size of the inner surface is expanded to 3.39 μm as in this comparative example, the average depth of the pores on the inner surface becomes deeper and blood compatibility decreases. Therefore, it can be seen that it is preferable to control the average depth of the pores in the inner surface to be shallow.

Using the obtained hollow fiber membrane, the hollow fiber membrane module 7 was obtained by the above method (1). The hollow fiber membrane module 7 was measured for latex particle permeability at 0.04 μm and 0.06 μm, and the results showed that the permeability was 91% and 56%, respectively, and no permeation of particles having a particle size of 0.15 μm was observed. When measurement of vesicle permeability was performed using phosphatidylserine and CD9 as surface markers, the vesicle permeability was 0.3%. That is, the hollow fiber membrane of the present Comparative Example had insufficient vesicle permeability even when the surface pore size was large.

### Example 4

1056 hollow fiber membranes obtained in Example 2 were put in a cylindrical case having an inner diameter of 2.1 cm and a length of 31 cm. A nozzle was provided at a position of 1.5 cm with respect to the end surface length from each of both end surfaces of the cylindrical case. Next, the cylindrical case was set in a centrifuge, 5 mL of urethane resin (potting material) was injected from each of two nozzles to both ends, and the potting material was cured by rotating at 60 G for 15 minutes. After 15 minutes, 10 mL of the potting material was further injected from each of the two nozzles, and the potting material was again rotated at 60 G for 15 minutes to cure the potting material. After the two times of potting, the potting material on the end surface of the case was cut, and a nozzle was attached to prepare a hollow fiber membrane module 8. In the obtained hollow fiber membrane module 8, the effective length of the hollow fiber membrane was 29.0 cm, the membrane area was 0.3 m², and the filling rate was 49.6%.

500 mL of commercially available human serum was caused to flow from a nozzle at the end surface of the hollow fiber membrane module 8 to a nozzle located at a position of 1.5 cm with respect to the end surface length from the opposite end surface, and filtered. This filtration was performed as constant pressure filtration in which the liquid delivery pressure was constant at 1 kPa. In this instance, the nozzle attached to the case end surface is the liquid to be treated inlet, and the nozzle located at a position of 1.5 cm from the case end surface with respect to the end surface length is the filtrate outlet. This configuration is a configuration in which the human serum, which is the liquid to be treated, flows from the inside to the outside of the hollow fiber membrane and flows out from the filtrate outlet.

The vesicle concentration in the human serum before filtration and the vesicle concentration in the filtrate were each measured by the method described above using PS/CD9 as a marker, and the vesicle permeability was measured from the obtained values. As a result, the permeability was 99%. The time required for filtration was 38 minutes and 28 seconds.

It was confirmed that the module having the dimensions of hollow fiber membrane module 8 also had a high function as a module for removing the vesicle. A module of this dimension is sufficient in volume and easy to handle for use in human therapy.

### Example 5

The entire membrane of the hollow fiber membrane module 8 prepared in Example 4 was coated with a vinylpyrrolidone/vinyl propanoate random copolymer in the same manner as in Example 3, and irradiated with a γ-ray of 25 kGy to obtain a hollow fiber membrane module 9. An experiment in which human serum was subjected to constant pressure filtration in the same manner as in Example 4 using the hollow fiber membrane module 9 was conducted, and the result showed that the permeability of the vesicle was 99%, and the time required for filtration was 27 minutes and 42 seconds. The time required for filtration was reduced by 28% as compared to Example 4. This is considered to be because the vinylpyrrolidone/vinyl propanoate random copolymer supported on the separation membrane reduced the adhesion of proteins contained in human serum to the separation membrane and the resulting narrowing of the pores. That is, it is found that, when a hydrophilic polymer, particularly a biocompatible polymer such as a vinylpyrrolidone/vinyl propanoate random copolymer is supported on the separation membrane, not only the increase in pressure is suppressed in the constant speed filtration as in Example 3, but also the filtration time is shortened in the constant pressure filtration as in this Example, which is preferable.

TABLE 1

| | | | | Example 1 | Example 2 | Comparative Example 1 |
|---|---|---|---|---|---|---|
| Main component of hollow fiber membrane | | | | Polysulfone | Polysulfone | Polysulfone |
| Hydrophilic polymer | | | | Polyvinylpyrrolidone | Polyvinylpyrrolidone | Polyvinylpyrrolidone |
| Content (mass %) of hydrophilic polymer | | | | 13.5 | 12.8 | 11.5 |
| Proportion (mass %) of insoluble component | | | | 8.9 | 7.9 | 5.8 |
| Biocompatible Polymer | | | | — | — | — |
| Particle permeability (%) | | 0.15 | μm | 86 | 99 | 7 |
| | | 0.20 | μm | 69 | 68 | 1 |
| | | 1.00 | μm | 1 | 1 | 1 |
| Water permeability (mL/h/mmHg/m²) | | | | 20458 | 23907 | 11145 |
| Carboxylic acid ions detected by TOF-SIMS | | | | — | — | — |
| measurement | | | | | | |
| Cross-sectional structure of hollow fiber | | | | Asymmetric | Asymmetric | Asymmetric |
| membrane | | | | | | |
| Presence position of dense layer | | | | Closer to surface | Closer to surface | Closer to surface |
| Thickness of dense layer (μm) | | | | 4.5 | 4.3 | 7.3 |
| Inner diameter of hollow fiber membrane | | | | 317 | 313 | 300 |
| (μm) | | | | | | |
| Hollow fiber membrane thickness (μm) | | | | 50 | 71 | 80 |
| Average pore size (μm) of | | Inner | | 1.20 | 1.71 | 0.70 |
| surface of hollow fiber | | surface | | | | |
| membrane | | Outer | | 0.81 | 0.66 | 0.46 |
| | | surface | | | | |
| Open pore ratio (%) of | | Inner | | 27.7 | 22.2 | 19.9 |
| surface of hollow fiber | | surface | | | | |
| membrane | | Outer | | 32.7 | 28.2 | 24.8 |
| | | surface | | | | |
| Vesicle | Vesicle surface marker used | | | CD9/CD63 | CD9/CD63 | CD9/CD63 |
| permeability | for quantification | | | | | |
| | Vesicle | 30 | minutes | 100 | 95 | 60 |
| | permeability | 120 | minutes | 75 | 98 | 45 |
| | (%) | | | | | |
| | Vesicle surface marker used | | | PS/CD9 | PS/CD9 | PS/CD9 |
| | for quantification | | | | | |
| | Vesicle | 30 | minutes | — | 100 | — |
| | permeability | 120 | minutes | — | 99 | — |
| | (%) | | | | | |
| | Filtration side | 30 | minutes | 0.4 | 0.3 | 0.1 |
| | pressure change | 120 | minutes | −0.5 | −1.3 | −7.2 |

amount (kPa)

| | | | | Comparative Example 2 | Example 3 |
|---|---|---|---|---|---|
| Main component of hollow fiber membrane | | | | Polyethylene | Polysulfone |
| Hydrophilic polymer | | | | — | Polyvinylpyrrolidone |
| Content (mass %) of hydrophilic polymer | | | | — | 13.7 |
| Proportion (mass %) of insoluble component | | | | — | 9.8 |
| Biocompatible Polymer | | | | Ethylene/vinyl | Vinyl propionate/ |
| | | | | alcohol copolymer | vinylpyrrolidone |
| | | | | | copolymer |
| Particle permeability (%) | | 0.15 | μm | 30 | 80 |
| | | 0.20 | μm | 1 | 60 |
| | | 1.00 | μm | 1 | 1 |
| Water permeability (mL/h/mmHg/m²) | | | | 6295 | 19624 |
| Carboxylic acid ions detected by TOF-SIMS | | | | — | Vinyl propionate |
| measurement | | | | | |
| Cross-sectional structure of hollow fiber | | | | Uniform | Asymmetric |
| membrane | | | | | |
| Presence position of dense layer | | | | — | Closer to surface |
| Thickness of dense layer (μm) | | | | — | 4.3 |
| Inner diameter of hollow fiber membrane | | | | 350 | 317 |
| (μm) | | | | | |
| Hollow fiber membrane thickness (μm) | | | | 42 | 50 |
| Average pore size (μm) of | | Inner | | 0.44 | 1.18 |
| surface of hollow fiber | | surface | | | |
| membrane | | Outer | | 0.39 | 0.79 |
| | | surface | | | |
| Open pore ratio (%) of | | Inner | | 19.6 | 26.5 |
| surface of hollow fiber | | surface | | | |
| membrane | | Outer | | 17.0 | 31.8 |
| | | surface | | | |
| Vesicle | Vesicle surface marker used | | | CD9/CD63 | CD9/CD63 |
| permeability | for quantification | | | | |
| | Vesicle | 30 | minutes | 50 | 94 |
| | permeability | 120 | minutes | 58 | 90 |
| | (%) | | | | |
| | Vesicle surface marker used | | | PS/CD9 | PS/CD9 |
| | for quantification | | | | |
| | Vesicle | 30 | minutes | 31 | — |
| | permeability | 120 | minutes | 26 | — |
| | (%) | | | | |
| | Filtration side | 30 | minutes | −1.7 | 0.4 |
| | pressure change | 120 | minutes | −11.9 | 0.2 |

amount (kPa)

1. A hollow fiber membrane, comprising particles having a particle size of 0.15 μm have a permeability of 50% or more and 100% or less, and an average pore size of an inner surface of the hollow fiber membrane is 0.50 μm or more and 3.00 μm or less.

2. The hollow fiber membrane according to claim 1, wherein a thickness of a dense layer in a cross section of the hollow fiber membrane is 1.0 μm or more and 10.0 μm or less.

3. The hollow fiber membrane according to claim 1, wherein an average pore size of an outer surface of the hollow fiber membrane is 0.50 μm or more and 10.00 μm or less.

4. The hollow fiber membrane according to claim 1, wherein the hollow fiber membrane has an inner diameter of 150 μm or more and 500 μm or less and a membrane thickness of 20 μm or more and 100 μm or less.

5. The hollow fiber membrane according to claim 1, comprising:
a polysulfone-based polymer.

6. The hollow fiber membrane according to claim 1 comprising:
a hydrophilic polymer,
wherein a content of the hydrophilic polymer is 5.0 mass % or more and 15.0 mass % or less.

7. The hollow fiber membrane according to claim 1, wherein at least one of the hydrophilic polymers is a hydrophilic polymer containing a monocarboxylic acid vinyl ester unit as a repeating unit.

8. A hollow fiber membrane, which has permeability of a vesicle detectable by antibodies of phosphatidylserine and CD9 is 50% or more and 100% or less, and is used for separating the vesicle from a biological component.

9. The hollow fiber membrane according to claim 1, which is used for blood purification.

10. A hollow fiber membrane module comprising:
the hollow fiber membrane according to claim 1.

11. The hollow fiber membrane module according to claim 10, which is used for blood purification.

12. A vesicle-containing solution purified by the hollow fiber membrane module according to claim 10.

13. The hollow fiber membrane according to claim 8, which is used for blood purification.

14. A hollow fiber membrane module comprising:
the hollow fiber membrane according to claim 8.

15. The hollow fiber membrane module according to claim 14, which is used for blood purification.

16. A vesicle-containing solution purified by the hollow fiber membrane module according to claim 14.
